(19) United States
(12) Patent Application Publication
(10) Pub. No.: US 2018/0059127 A1
(43) Pub. Date: Mar. 1, 2018

(54) SKIN LIPIDOMIC ASSAY

(71) Applicant: Oregon State University, Corvallis, OR (US)

(72) Inventors: Arup Indra, Corvallis, OR (US); Gitali Ganguli-Indra, Corvallis, OR (US); Shan Li, Corvallis, OR (US); Jaewo Choi, Corvallis, OR (US)

(21) Appl. No.: 15/565,366

(22) PCT Filed: Apr. 8, 2016
(86) PCT No.: PCT/US16/26751

§ 371 Date: Oct. 9, 2017

Related U.S. Application Data
(60) Provisional application No. 62/146,179, filed on Apr. 10, 2015.

(51) Int. Cl. G01N 33/92; A61K 31/575; A61K 9/00; A61K 31/20; A61K 31/164; A61K 31/23

(52) U.S. Cl. CPC G01N 33/92; A61K 31/575; A61K 31/23; A61K 31/20; A61K 31/164; A61K 9/0014

(57) ABSTRACT

A method for determining a lipid imbalance in a subject is provided. The method includes providing one or more tape strips comprising a skin surface sample obtained from the subject; extracting epidermal lipids; detecting a composition of lipids present in the extracted sample; and comparing the composition of lipids to a control. A difference in the composition of lipids as compared to the control identifies the lipid imbalance in the subject. A method of augmenting a lipid deficiency in skin of a subject, for example to treat or inhibit infection, prevent water loss, improve hydration and restore barrier is also provided. The method includes identifying a deficiency in one or more lipids in a skin sample obtained from the subject; formulating a topical therapeutic composition that contains the one or more lipids, a similar lipid, or a subset thereof; and providing the composition to the subject. Topical formulations for augmenting a lipid deficiency in the skin of a subject are also disclosed.

### CROSS REFERENCE TO RELATED APPLICATION

This application claims the priority benefit of the earlier filing date of U.S. Provisional Patent Application No. 62/146,179, filed Apr. 10, 2015, which is specifically incorporated by reference herein in its entirety.

### STATEMENT OF GOVERNMENT INTEREST

This invention was made with government support under contract/grant nos: HHSN272201000020C, HHSN272201000017C, UM2AI117870, and U19AI117673-01 awarded by the National Institute of Allergy and Infectious Diseases, National Institutes of Health, Department of Health and Human Services. The government has certain rights in the invention.

### TECHNICAL FIELD

The present disclosure relates to the field of molecular biology and, more particularly, to a method for diagnosing, monitoring, and treating a subject suffering from a skin disorder.

### BACKGROUND

All mammals develop a critically important epidermal permeability barrier (EPB) in utero as an essential outcome of the skin's differentiation program. In infants, failure to construct a competent EPB is a potentially life-threatening problem. A compromised EPB leads to enhanced percutaneous absorption of harmful chemicals, as well as increased trans-epidermal water loss, thereby contributing to dehydration, poor thermoregulation, and fragile skin. Barrier defects also contribute to hereditary and acquired chronic inflammatory skin disorders, such as psoriasis and atopic dermatitis (AD). In 2012, approximately fifteen million Americans were estimated to suffer from atopic dermatitis, which accounts for approximately 10-20% of all visits to dermatologists and over $1 billion in estimated healthcare costs annually.

Glucocorticoids are currently the most prescribed drugs for treatment of AD and have long been used to reduce skin inflammation. However, over time administration of glucocorticoids themselves can deteriorate epidermal barrier function by causing skin thinning. Further, while the pharmaceutical industry has successfully marketed more potent glucocorticoids, it has not, to date, successfully developed drugs that can be used to effectively improve epidermal barrier function. Thus, for both humanitarian and economic reasons, it is important to develop new and effective treatments for these skin barrier defects and associated inflammatory skin diseases, and to delineate the underlying molecular mechanisms by which therapeutic interventions help to improve health of affected patients.

### BRIEF DESCRIPTION OF THE DRAWINGS

Embodiments will be readily understood by the following detailed description in conjunction with the accompanying drawings and the appended claims. Embodiments are illustrated by way of example and not by way of limitation in the figures of the accompanying drawings.

FIG. 1 is a flow diagram illustrating steps for the isolation and characterization of Stratum corneum (SC) lipids from a subject.

FIGS. 2A and 2B are a mass spectra illustrating representative LC MS/MS data of the detected peaks for lipids in a normal versus atopic dermatitis (AD) subjects.

FIGS. 3A and 3B are graphs of representative LC MS/MS data of the detected peak for a specific long chain ceramide [EOS]C70 in a normal subject (3A) and AD subject (3B).

FIGS. 4A and 4B are a bar graph and plot illustrating the relative levels of saturated ceramides in normal and AD subjects after normalization with internal standards.

FIG. 5 is a bar graph illustrating the relative levels of unsaturated ceramides in normal and AD subjects after normalization with internal standards.

FIGS. 6A-6D are a set of plots illustrating a statistically significant increase in SC unsaturated ceramides in AD subjects.

FIGS. 7A-7D are a set of plots illustrating altered SC unsaturated ceramides in AD subjects. Circled AD subgroups have reduced levels of unsaturated ceramides.

FIGS. 8A and 8B are a bar graph and plot illustrating altered SC sphingosine in a selected subgroup of AD subjects.

FIGS. 9A-9D are bar graphs and plots illustrating altered SC free fatty acids (FFA) in a selected subgroup of AD subjects.

FIGS. 10A-10D are a set of plots illustrating the average SC FFA distribution in AD subjects.

FIGS. 11A-11C are a bar graph and plots illustrating altered SC cholesterol and cholesterol-sulfate in an AD subgroup.

FIGS. 12A-12F are a set of bar graphs and plots illustrating lipid content differences between AD-S. Aureus− and AD-S. Aureus+ subjects.

FIGS. 13A-13F are a set of bar graphs and plots illustrating lipid content differences between AD-S. Aureus− and AD-S. Aureus+ subjects.

FIGS. 14A-14D is a set of plots illustrating basal TEWL, serum TARO, IgE levels and eosinophil counts in healthy non-atopic (NA), AD-S. Aureus− and AD-S. Aureus+ subjects. Boxplots show increased basal TEWL (FIG. 14A), serum TARO (FIG. 14B), IgE levels (FIG. 14C) and eosinophil counts (FIG. 14D) in all AD subjects including AD-S. Aureus−, AD-S. Aureus+ patients and healthy individuals. Significant differences were observed between AD-S. Aureus− and AD-S. Aureus+ for serum TARO and IgE level. One subject with an extremely a low value (0.) of TARO and another with a low value (0.) of eosinophil counts in AD-S. Aureus− group possibly due to a technical error, have been removed. All data were log-transformed and adjusted for age and gender. *P<0.05, **P<0.01, ***P<0.001.

### DETAILED DESCRIPTION

In the following detailed description, reference is made to the accompanying drawings that form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.). The terms “patient” and “subject” are used interchangeably herein and includes human and non-human animals. In one example, the patient or subject is a mammal, such as a human.

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In addition, the materials, methods, and examples are illustrative only and not intended to be limiting.

### Description of Several Embodiments

Atopic dermatitis (AD) is a chronic inflammatory skin disease characterized by disrupted epidermal barrier functions. Staphylococcus aureus (S. aureus) infection aggravates AD. Epidermal stratum corneum (SC) comprises corneocytes and a lipid-enriched extracellular matrix. Lipid constituents of SC include ceramides (CERs), free fatty acids (FFAs), cholesterol and others including triglycerides (TGs). Lipids are key players in skin barrier maintenance and help the body retain moisture for hydration and help protect bodies against external irritation and infection. However, the lipid composition of every individual subject is slightly different from others. Prior to this disclosure there was no standard tool or methodology for detecting changes or abnormalities in the lipid composition of individual subjects. In addition, methods of ameliorating such changes or abnormalities were lacking.

Using the power of lipidomics, the inventors have measured the lipids in human skin to create lipid profiles that characterize imbalances in the composition of lipids present in a subject's skin. The inventors have compared the lipid metabolome of subjects with skin conditions, including atopic dermatitis and/or S. aureus colonization, to that of healthy controls. Such lipidomic signatures, or lipid profiles, facilitate the determination of which subjects will benefit from a particular therapy, for example, lipid add-back therapy, and provide the tools for a personalized approach to such therapy.

Metabolomics is the study of metabolism at the global level. It involves systematic study of the metabolome, the complete repertoire of small molecules present in cells, tissues or organisms. A subset of Metabolomics is lipidomics, which refers to the use of metabolomics as applied to the evaluation of lipid metabolites in biological samples, such as skin. Lipid profiling generally involves an evaluation of lipid metabolites in one or more lipid classes (e.g., free fatty acids, triglycerides, cholesterols, and ceramides).

The inventors have established that altered lipid composition of the skin of a subject can lead to impaired protective barrier functions, which can lead to the onset and progression of skin inflammation and eczema as well as susceptibility to infection, such as S. aureus infection. This altered, or abnormal, lipid composition of the skin can lead to impaired protective barrier functions, which can lead to the onset and progression of skin inflammation and eczema, as well as bacterial colonization, such as S. aureus colonization. Thus, measuring skin lipid composition provides a tool or methodology for detecting changes in the lipid composition in different individuals or subjects (e.g. cats, dogs, humans, etc.), who are either susceptible to or have an inflammatory skin disease (e.g. eczema, or psoriasis).

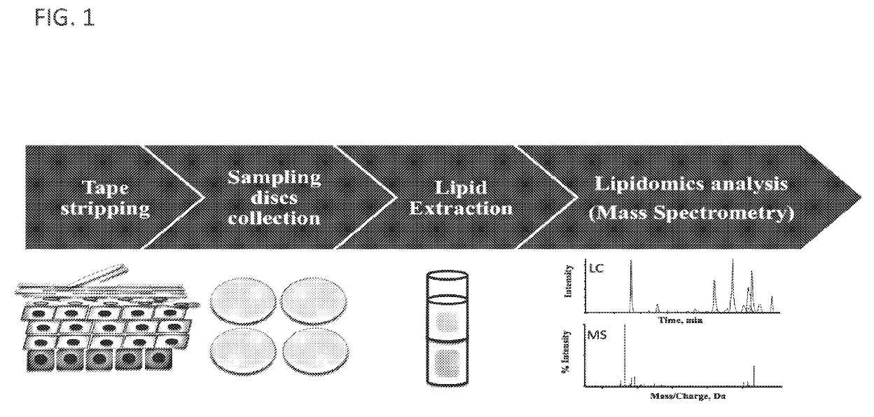

The inventor's unexpected discovery demonstrates that altered lipid composition in the skin of eczema patients may be a determinant of their disease severity and causally related to the onset and progression of the disease. The inventors have further discovered that measuring skin lipid composition provides a fast, reliable, reproducible, and non-invasive tool to detect onset of AD pathogenesis and characterize AD subtypes in humans. Furthermore, analyzing an individual subject's skin lipid composition can be used to tailor personalized medicine technologies, such as personalized treatments, therapies, and compositions to a subject. The disclosed methods may provide a simple one-step method to isolate epidermal lipids of normal, atopic dermatitis (AD), or eczema subjects in a non-invasive way, to provide for isolated skin surface lipids. In embodiments, the methods are a fast, simple, and reliable one-step way for the isolation of skin lipids from a limited number corneocytes, for example as retrieved by a non-invasive tape stripping method. This standardized method can be used to characterize the skin lipids via untargeted lipidomics using, for example, a LC-MS/MS multiple reaction monitoring (MRM) technique. An example method is shown in FIG. 1.

Disclosed is a method for determining a lipid imbalance in the skin of the subject, for example an imbalance in the lipid profile of the subject's skin. The disclosed method includes obtaining, or providing, one or more skin samples of a subject, for example, providing one or more tape strips that include a skin surface sample obtained from the subject. Skin samples include cells and/or lipids of the stratum corneum. Tape stripping is a non-invasive and fast method for stratum corneum (SC) sample collection. The tape strips are typically square or circular need to be of the same size and area and have to be applied with the help of the same pressure instrument. The lipids are extracted from the skin samples and the composition of lipids present in the extracted sample is detected. This detected composition of lipids in the sample provides a lipid profile of the subjects skin that is used to determine if the subject's skin has a lipid imbalance, for example relative to the amount and/or class, type, or subtype, of lipids present in the skin of a normal subject.

In embodiments, the composition of lipids, or lipid profile, is compared to a control. A difference in the composition of lipids as compared to the control identifies the lipid imbalance in the subject. An imbalance can be an increase in a certain lipid, or lipids. Conversely a lipid imbalance can be a decrease in a certain lipid, or lipids, or even an increase in some lipids and a decrease in others. In embodiments, the change detected is an increase or decrease in the level of a lipid as compared to a control, such as a reference value or a healthy control subject. Controls or standards for comparison to a sample, include samples believed to be normal as well as laboratory values (e.g., range of values), even though possibly arbitrarily set, keeping in mind that such values can vary from laboratory to laboratory. Laboratory standards and values can be set based on a known or determined population value and can be supplied in the format of a graph or table that permits comparison of measured, experimentally determined values. A control can be a sample or standard used for comparison with a test sample, such as a sample obtained from a subject or patient (or plurality of patients). In some embodiments, the control is a sample obtained from a healthy patient (or plurality of patients) (also referred to herein as a “normal” control). In some embodiments, the control is a historical control or standard value (e.g. a previously tested control sample or group of samples that represent baseline or normal values, such as baseline or normal values in a normal subject or subject). In some examples the control is a standard value representing the average value (or average range of values) obtained from a plurality of patient samples (such as an average value or range of values of lipids in the skin of a normal patient).

In some embodiments, the lipid imbalance is diagnostically significant change in one or more lipids. As used herein a “diagnostically significant change” refers to an increase or decrease in the level of one or more lipids in a biological sample that is sufficient to allow one to distinguish one patient population from another (such as a subject suffering DA from one that is not). In some embodiments, the diagnostically significant change is at least 2-fold, at least 3-fold, at least 4-fold, at least 5-fold, at least 6-fold, at least 8-fold, at least 10-fold, at least 15-fold, at least 20-fold, at least 30-fold, or at least 40-fold relative to a control. In some examples, the detected increase or decrease is an increase or decrease of at least 2-fold compared with the control or standard.

The lipid profile may be used as a quantitative trait to identify different sub-types and sub-groups of AD (e.g. eczema) and to design subject specific (personalized) formulations. For example, emollients that include at least one lipid that has been found to be deficient in a characterized subject or a similar lipid, for example that complements the deficiency. The lipid profile can be used to design formulations of emollients (creams, lotions, or other delivery methods) with specific lipid compositions that can stabilize an abnormal lipid composition in an individual such as an AD positive individual, to prevent onset of AD (e.g. eczema) in susceptible individuals, and to mitigate disease progression in affected individuals, for example progression of AD to more advance disease such as eczema and/or infection with S. aureus.

As used herein, the term “lipid” can refer to a single species within a lipid class, a subset of species within a lipid class, or the entire lipid class. “Lipid” is intended broadly and encompasses a diverse range of molecules that are relatively water-insoluble or nonpolar compounds of biological origin, including waxes, triglycerides, free fatty acids, triglicerides, diacylglyercols, fatty-acid derived phospholipids, sphingolipids, such as ceramides, glycolipids and terpenoids, such as retinoids, cholesterol, cholesterol esters, and steroids. Some lipids are linear aliphatic molecules, while others have ring structures.

A lipid “class” refers to a collection of lipid molecules that share structural and/or biochemical properties. Accordingly, lipids within any class(es) can be evaluated. Suitable lipid classes include polar and non-polar classes of lipids. Exemplary non-polar lipid classes include without limitation the free fatty acids, monoacylglycerides, diacylglycerides, triacylglycerides, sterols and/or cholesterol esters. Exemplary polar classes include without limitation the phospholipid classes such as phosphatidic acid, lysophosphatidylcholine, sphingomyelin, phosphatidylinositol, phosphatidylglycerol, phosphatidylcholine, phosphatidylserine, phosphatidylethanolamine, lysophosphatidylethalolamine, cardiolipin and/or lysocardiolipin and phospholipid precursors such as ceramides.

Fatty acids are unbranched hydrocarbon chains, connected by single bonds alone (saturated fatty acids) or by both single and double bonds (unsaturated fatty acids). Examples of saturated fatty acids include but are not limited to butyric acid, lauric acid, myristic acid, pentadecanoic acid, palmitic acid, stearic acid, arachidic acid, behenic acid and lignoceric acid. Examples of unsaturated fatty acids include but are not limited to linolenic acid, docosahexaenoic acid, eicosapentaenoic acid, linoleic acid, arachidonic acid, oleic acid, and erucic acid. Particular classes of fatty acids include omega-3 fatty acids (e.g., alpha-linolenic, stearidonic, eicosatrienoic, eicosatetraenoic, eicosapentaenoic, docosapentaenoic, docosahexaenoic and tetracosahexaenoic acids), omega-6 fatty acids (e.g., linoleic, gamma-linolenic, eicosadienoic, homo-gamma-linolenic, arachidonic, docosadienoic, docosatetraenoic and 4,7,10,13,16-docosapentaenoic acids) and omega-9 fatty acids (e.g., myristoleic, palmitoleic, vaccenic, oleic, eicosenoic, mead, erucic and nervonic acids). Other fatty acids include plasmalogen-linked fatty acids including but not limited to plasmalogen 16:0, plasmalogen 18:0, plasmalogen 18:1n7 and plasmalogen 18:1n9. Other fatty acids include but are not limited to palmitelaidic acid, elaidic acid, 8-eicosaenoic acid and 5-eicosaenoic acid. All of the above can be detected with the disclosed methods, provided they are in an analyzed skin sample.

Ceramides (CER) are a family of waxy lipid molecules. A ceramide is composed of sphingosine and a fatty acid. Ceramides are found in high concentrations within the cell membrane of cells. They are one of the component lipids that make up sphingomyelin, one of the major lipids in the lipid bilayer. Particular classes of ceramides include CER [EOdS], CER [EOS], CER [EOP], CER [EOH], CER [OdS], CER [OS], CER [OP], CER [OH], CER [NdS], CER [NS], CER [NP], CER [NH], CER [AdS], CER [AS], CER [AP], CER [AH], and CER [EO]. All of the above can be detected with the disclosed methods, provided they are in an analyzed skin sample.

Triglycerides (TG, triacylglycerol, TAG, or triacylglyceride) are esters derived from glycerol and three fatty acids (tri−+ glyceride). Triglycerides are the main constituent of body fat in humans and animals, as well as vegetable fat. There are many different types of triglyceride, with the main division being between saturated and unsaturated types. Saturated fats are “saturated” with hydrogen—all available places where hydrogen atoms could be bonded to carbon atoms are occupied. These have a higher melting point and are more likely to be solid at room temperature. Unsaturated fats have double bonds between some of the carbon atoms, reducing the number of places where hydrogen atoms can bond to carbon atoms. These have a lower melting point and are more likely to be liquid at room temperature. All of the above can be detected with the disclosed methods, provided they are in an analyzed skin sample.

Analysis of the fatty acid class or fatty acid moieties incorporated into lipids of other classes can evaluate any characteristic including but not limited to chain length, the degree of saturation/desaturation and/or the position of any double-bond(s) that are present. With respect to chain length, the lipid profile can evaluate the presence of short- (less than 8 carbons), medium- (8 to 14 carbons), long- (e.g., 14 to 18 carbons) and very long- (e.g., 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88 or more carbons) fatty acids, optionally with a further evaluation of saturation/desaturation. For example, in some embodiments saturated fatty acids are detected. In other embodiments, mono- and/or poly- (i.e., two or more unsaturated bonds) unsaturated fatty acids are evaluated. The position of the unsaturated bond(s) can also be evaluated, for example, omega-3 (i.e., n3), omega-6 (i.e., n6) and/or omega-9 (i.e., n9) fatty acids have double-bonds in the 3, 6 or 9 position, respectively. Further, the presence of cis or trans bonds within unsaturated fatty acids can be assessed. In particular embodiments the lipid profile includes a lipid that comprises a fatty acid moiety, such as a ceramide or triglyceride. In some embodiments, the diagnostic and/or prognostic lipid profile can comprise one or more free fatty acids. As a further option, the lipid profile can evaluate specific free fatty acids and/or fatty acid components within one or more lipid classes. Free fatty acids and fatty acid moieties that can be assessed in the lipid profile include but are not limited to: 14:0, 15:0, 16:0, 16:1, 18:0, 18:1, 18:2, 20:0, 22:0, 24:0, 38:0, 40:0, 46:1, 48:0, 48:2, 50:1, 50:2, 50:3, 52:0, 54:0, 58:2, 66:0, 68:0, 70:0, 14:1n5, 16:1n7, 18:1n7, 18:1n9, 20:1n9, 20:3n9, 22:1n9, 24:1n9, 18:2n6, 18:3n6, 14:1n5, 20:1n15, 20:1n12, 18:3n3, 18:4n3, 20:3n3, 20:4n3, 20:5n3, 22:5n3, 22:6n3, 24:6n3, 18:2n6, 24:6n3, 18:2n6, 18:3n6, 20:2n6, 20:3n6, 20:4n6, 22:2n6, 22:4n6, 22:5n6, t16:1n7, t18:1n9, t18:2n6, dm16:0, dm18:0, dm18:1n9, dm18:1n7, total saturated fatty acids, total monounsaturated fatty acids, total polyunsaturated fatty acids, total LC fatty acids, total n3 (omega 3) fatty acids, total n6 fatty acids, total n7 fatty acids, total n9 fatty acids, and/or total dm fatty acids. Further, the lipid profile can evaluate without limitation tetradecanoic acid, pentadecanoic acid, hexadecanoic acid, heptadecanoic acid, octadecanoic acid, eicosanoic acid, docosanoic acid, tetracosanoic acid, 9-tetradecenoic acid, 9-hexadecenoic acid, 11-octadecenoic acid, 9-octadecenoic acid, 11-eicosenoic acid, 5,8,11-eicosatrienoic acid, 13-docosenoic acid, 15-tetracosenoic acid, 9,12,15-octadecatrienoic acid, 6,9,12,15-octadecatetraenoic acid, 11,14,17-eicosatrienoic acid, 8,11,14,17-eicosictetraenoic acid, 5,8,11,14,17-eicosapentaenoic acid, 7,10,13,16,19-docosapentaenoic acid, 4,7,10,13,16,19-docosahexaenoic acid, 6,9,12,15,18,21-tetracoshexaenoic acid, 9,12-octadecadienoic acid, 6,9,12-octadecatrienoic acid, 11,14-eicosadienoic acid, 8,11,14-eicosatrienoic acid, 5,8,11,14-eicosicatetraenoic acid, 13,16-docsadienoic acid, 7,10,13,16-docosicatetraenoic acid, 4,7,10,13,16-docosapentaenoic acid, 9-trans-hexadecenoic acid, 9-trans-octadecenoic acid, 8-eicosaenoic acid, 5-eicosaenoic acid, plasmalogen fatty acids, 5b-cholestan-3b-ol, 5a-cholestan-3b-ol, 5-cholesten-3b-ol, 5,24-cholestadien-3b-ol, 5-cholestan-25a-methyl-3b-ol, 5-cholestan-24b-methyl-3b-ol, 5-cholesten-24b-ethyl-3b-ol, and/or 5,22-cholestadien-24b-ethyl-3b-ol, each as a free fatty acid or a fatty acid moiety incorporated into a larger lipid molecule such as ceramides and triglycerided. Thus, those skilled in the art will appreciate that the lipid profile can evaluate any combination of the foregoing characteristics of fatty acids (e.g., ratios, chain length, saturation/desaturation and/or position of any double-bonds), whether present in free fatty acids or fatty acid moieties incorporated into larger lipid molecules in other lipid classes. It is intended that the lipid profile can evaluate free fatty acids and fatty acid moieties that are incorporated into lipid molecules within other lipid class(s) having any combination of features described herein such as lipid class, chain length, saturation/desaturation and/or position of any double-bond(s) as if the individual species embodying the various combinations of features.

Example of lipids that can be detected and included in a lipid profile include the ceramides CER[AH]C38, CER[AH]C48, CER[AP]C40, CER[NDS]C52, CER[NDS]C54, CER[EOH]C66, CER[EOH]C68, CER[EOS]C70, the free fatty acids FFA16:1 and FFA18:1; the triglycerides TG46:1, TG48:1, TG48:2, TG50:1, TG50:2, TG50:3, TG58:2. Additional lipids, and lipid classes that can be evaluated and/or detected in skin samples include those described in Masukawa et al. J Lipid Res. 2009 August; 50(8):1708-19; van Smeden et al., Exp Dermatol. 2014 January; 23(1):45-52; Smeden et al., J Lipid Res. 2011 June; 52(6):1211-21; and Janssens et al., J Lipid Res. 2012 December; 53(12):2755-66, each of which is specifically incorporated herein by reference. Other example of lipids that can be detected and include in a lipid profile include sterols, such as cholesterol and cholesterol-sulfate, and triglycerides.

A “lipid profile,” as used herein, refers to the evaluation of one or more lipids within a biological sample. In particular embodiments, 2 or more, 3 or more, 4 or more, 5 or more, 6 or more, 7 or more, 8 or more, 9 or more, 10 or more, 12 or more, 15 or more, 20 or more, 50 or more, 100 or more, or an even greater number of lipids are evaluated, such as from 1 to 200, from 50 to 175, from 75 to 125, or about 100 skin lipids including (saturated and un-saturated ceramides, free fatty acids, cholesterol, cholesterol-sulfate, triglycerides, sphingosine and sphinganine). The characterized lipids may be isolated skin surface lipids. In embodiments wherein 2 or more lipids are assessed, the 2 or more lipids can belong to the same class or can be belong to 2 or more, 3 or more, 4 or more, 5 or more, 6 or more, 7 or more or a greater number of different lipid classes. The lipid profile can be quantitative, semi-quantitative and/or qualitative. For example, the lipid profile can evaluate the presence or absence of a lipid, can evaluate the presence of a lipid(s) above or below a particular threshold, and/or can evaluate the relative or absolute amount of a lipid(s). Not all lipids in a sample need be evaluated for a lipid profile.

In representative embodiments, the lipid profile provides a compositional analysis in which 2 or more, 3 or more, 4 or more, 5 or more, 6 or more, 7 or more, 8 or more, 9 or more, 10 or more, 12 or more, 15 or more, 10 or more, 50 or more, 100 or more or a greater number of lipids are evaluated within a single class or within 2 or more, 3 or more, 4 or more, 5 or more, 6 or more, 7 or more or a greater number of different lipid classes. Further, the lipid profile can assess 2 or more, 3 or more, 4 or more, 5 or more, 6 or more, 7 or more or a greater number of different classes, and can evaluate 2 or more, 3 or more, 4 or more, 5 or more, 6 or more, 7 or more, 8 or more, 9 or more, 10 or more, 12 or more, 15 or more, 20 or more, 50 or more, 100 or more or a greater number of lipids within each class.

Optionally, the lipid profile provides a compositional analysis (e.g., mole percentage (%) of the lipid) within its class. For example, the lipid profile can include an evaluation (e.g., quantitation or determination of mole % within class) of 2 or more, 3 or more, 4 or more, 5 or more, 6 or more, 7 or more, 8 or more, 9 or more, 10 or more, 12 or more, 15 or more, 20 or more, 50 or more, 100 or more, or a lipids within one or more lipid classes (for example, saturated and un-saturated ceramides, free fatty acids, cholesterol, cholesterol-sulfate, triglycerides, sphingosine and sphinganine).

In some embodiments, the method further includes assigning the subject to a skin lipid deficiency category on the basis of the lipid imbalance in the subject, for example, so that a treatment can be provided to ameliorate the lipid imbalance. In embodiments, the subject is assigned to one of the following skin lipid deficiency categories: group I; group II, and group III. Group I includes FFA 16:1 and FFA 18:1 (see Table 1 and 2), both of which are reduced/deficient in highest percentage of AD patients (51%) without any significant changes in any other lipids. The group II category includes CER[AH]C48, CER[EOH]C66 and CER[EOH] C68 lipids (deficient in 30% of AD subjects without significant changes in lipids from Group I) in addition to the two lipids in Group I. This group is designated to treat AD subjects with more severe AD status and/or those non-responding well to treatment with Group I lipids. Also, the Group II lipids would cover a higher percentage of atopic patients. The Group III category includes CER[AP]C40, CER[NDS]C52, CER[NDS]C54, CER[AH]C48 and CER[EOS]C70 lipids in addition to all of the lipids in Groups I and II. This group is designated in order to treat AD subjects with most severe AD status and disease progressing to atopic asthma and allergic rhinitis (as part of the atopic march). All of the above lipids could be used in formulation after supplementation with emollient or cream base containing oil/water emulsion.

The disclosed methods may further be used along with transcriptomic, proteomic and GWAS analyses to identify the expression of lipid metabolizing genes. Thus, the disclosed methods may also be employed to determine an association of transcriptomic, proteomics and lipidomic data, for example to analyze/establish a relationship between altered lipid metabolism, barrier dysfunction, and AD pathogenesis.

The present disclosure provides for detection, measurement, characterization, or monitoring of skin lipid composition from mammals in a fast, reliable, reproducible, and non-invasive manner. In embodiments, the detection, measurement, characterization, or monitoring of skin lipid composition provides a tool to detect onset of inflammatory skin disease. For example, without limiting the invention, inflammatory skin diseases include AD eczema and psoriasis. The mammal may be any mammal, e.g., cat, dog, human, etc. In embodiments, the present disclosure provides for detection, measurement, characterization, or monitoring of skin lipid composition from mammals useful in characterizing different subtypes of AD, eczema, psoriasis, icthyosis, Netherton Syndrome in subjects, or any other skin diseases linked with epidermal barrier dysfunction and monitored by increased trans epidermal water loss (TEWL).

The subjects may be any mammal, including for example, cats, dogs, or humans. The detection, measurement, characterization, or monitoring of skin lipid composition can provide guidance and be employed in personalized medicine or therapy.

Those skilled in the art will appreciate that the lipid profile can be relatively straight-forward (e.g., detecting the presence, amount and/or mole % within class) of relatively few (e.g., one, two, three or four) lipids or can be quite complex and encompass tens or even hundreds of lipids, optionally including a compositional analysis of the metabolites within one or more lipid classes. Thus, it will also be apparent that the lipid profiles and the methods described herein can be practiced to evaluate any combination of the lipid characteristics described herein.

In some embodiments, the level of a lipid or multiple lipids is normalized against specific lipid internal standards. For example, the level of cholesterol sulfate can be normalized against an internal standard (e.g deuterium labeled cholesterol sulfate) or relative to the total protein isolated from the same tape strips, or for example, a lipid that is relatively stable in amount under a variety of conditions in the subject.

Quantitative lipid data include molar quantitative data, mass quantitative data and relational data by either moles or mass (mole % or weight %, respectively) for individual lipid or subsets of lipids. In some embodiments, quantitative aspects of lipidomic analysis can be provided and/or improved by including one or more quantitative internal standards during the analysis, for instance, one standard for each lipid class. Quantitative data can be integrated from multiple sources (e.g., the data do not need to be generated with the same assay, in the same location and/or at the same time) into a single seamless database regardless of the number of lipids measured in each, discrete, individual analysis.

The lipidomics profile can be based on quantitative, semi-quantitative and/or qualitative analysis. For example, qualitative methods can be used to detect the presence or absence of a lipid in a biological sample, such as an extracted skin sample. Semi-quantitative quantitative methods can be used to determine a level of a particular lipid above a threshold value or to determine ratios of different lipids, without assigning an absolute or relative numerical value. Quantitative methods can be used to determine a relative or absolute amount of a particular lipid in the biological sample, such as an extracted skin sample.

In semi-quantitative methods, a threshold or cutoff value can be determined by any means known in the art, and is optionally a predetermined value. In particular embodiments, the threshold value is predetermined in the sense that it is fixed, for example, based on previous experience with the assay and/or a population of affected and/or unaffected subjects. Alternatively, the term “predetermined” value can also indicate that the method of arriving at the threshold is predetermined or fixed even if the particular value varies among assays or may even be determined for every assay run.

The lipidomics analysis can generate high-density data sets that can be evaluated using informatics approaches. High data density informatics analytical methods are known and software is available to those in the art, e.g., cluster analysis (Pirouette, Informetrix), class prediction (SIMCA-P, Umetrics), principal components analysis of a computationally modeled dataset (SIMCA-P, Umetrics), 2D cluster analysis (GeneLinker Platinum, Improved Outcomes Software), and metabolic pathway analysis (biotech.icmb.utexas.edu). The choice of software packages offers specific tools for questions of interest (Kennedy et al., Solving Data Mining Problems Through Pattern Recognition. Indianapolis: Prentice Hall PTR, 1997; Golub et al., (1999) Science 286:531-7; Eriksson et al., Multi and Megavariate Analysis Principles and Applications: Umetrics, Umea, 2001). In general, any suitable mathematic analyses can be used to evaluate one, two or more lipids in a lipid profile. For example, methods such as multivariate analysis of variance, multivariate regression, and/or multiple regression can be used to determine relationships between dependent variables (e.g., clinical measures) and independent variables (e.g., levels of lipids). Clustering, including both hierarchical and nonhierarchical methods, as well as nonmetric Dimensional Scaling can be used to determine associations among variables and among changes in those variables.

In addition, principal component analysis is a common way of reducing the dimension of studies, and can be used to interpret the variance-covariance structure of a data set. Principal components may be used in such applications as multiple regression and cluster analysis. Factor analysis is used to describe the covariance by constructing “hidden” variables from the observed variables. Factor analysis may be considered an extension of principal component analysis, where principal component analysis is used as parameter estimation along with the maximum likelihood method. Furthermore, simple hypothesis such as equality of two vectors of means can be tested using Hotelling's T squared statistic.

In embodiments, extracting epidermal lipids from the skin surface sample includes contacting the one or more tape strips with an extraction solvent. In some embodiments, an extraction solvent comprises a mixture of a non-polar solvent, a polar solvent, such as polar protic solvent, and water, for example a mixture of chloroform (CHCl₃), methanol, and water. Examples of non-polar solvents include hexane, cyclo-hexane, toluene, 1,4-dioxane, chloroform, diethyl ether, and dichloromethane (DCM), among others. Examples of polar protic solvents include formic acid, n-butanol, isopropanol, nitromethane, ethanol, and methanol. In a particular embodiment, the ratio of chloroform, methanol, and water in the mixture is approximately 1:2:0.5, respectively, although other ratios can be used effectively.

In particular embodiments, the lipid profiles detect about 25% or more, about 40% or more, about 50% or more, about 60% or more, about 70% or more, about 75% or more, about 80% or more, about 85% or more, about 90% or more, about 95% or more, about 97% or more, about 98% or more, or about 99% or more of the lipids in a sample, such as a skin sample.

The lipid profile can be determined using any suitable method. The different classes of lipids and methods of detecting and optionally quantifying the same are well known in the art (e.g., thin layer chromatography, gas chromatography, liquid chromatography, mass and NMR spectrometry, and any combination thereof (e.g., GC/MS), and the like).

Mass spectrometry is particularly suited to the identification of lipids from biological samples, such as those descried herein. Typically, mass spectrometers generate gas phase ions from a sample (such as a sample containing lipids obtained from a skin sample). The gas phase ions are then separated according to their mass-to-charge ratio (m/z) and detected. Suitable techniques for producing vapor phase ions for use in the disclosed methods include without limitation electrospray ionization (ESI), matrix-assisted laser desorption-ionization (MALDI), surface-enhanced laser desorption-ionization (SELDI), chemical ionization, and electron-impact ionization (EI). Separation of ions according to their m/z ratio can be accomplished with any type of mass analyzer, including quadrupole mass analyzers (Q), time-of-flight (TOF) mass analyzers (for example linear or reflecting) analyzers, magnetic sector mass analyzers, 3D and linear ion traps (IT), Fourier-transform ion cyclotron resonance (FT-ICR) analyzers, and combinations thereof (for example, a quadrupole-time-of-flight analyzer, or Q-TOF analyzer).

In some embodiments, the mass spectrometric technique is tandem mass spectrometry (MS/MS) and the presence of a lipid from a skin sample is detected. Typically, in tandem mass spectrometry a lipid entering the tandem mass spectrometer is selected and subjected to collision induced dissociation (CID). The spectra of the resulting fragment ion is recorded in the second stage of the mass spectrometry, as a so-called CID spectrum. Suitable mass spectrometer systems for MS/MS include an ion fragmentor and one, two, or more mass spectrometers, such as those described above. Examples of suitable ion fragmentor include, but are not limited to, collision cells (in which ions are fragmented by causing them to collide with neutral gas molecules), photo dissociation cells (in which ions are fragmented by irradiating them with a beam of photons), and surface dissociation fragmentor (in which ions are fragmented by colliding them with a solid or a liquid surface). Suitable mass spectrometer systems can also include ion reflectors.

Prior to mass spectrometry the sample may be subjected to one or more dimensions of chromatographic separation, for example, one or more dimensions of gas, liquid, or size exclusion chromatography. Representative examples of chromatographic separation include paper chromatography, thin layer chromatography (TLC), liquid chromatography, column chromatography, fast protein liquid chromatography (FPLC), ion exchange chromatography, size exclusion chromatography, affinity chromatography, high performance liquid chromatography (HPLC), nano-reverse phase liquid chromatography (nano-RPLC), poly acrylamide gel electrophoresis (PAGE), capillary electrophoresis (CE), reverse phase high performance liquid chromatography (RP-HPLC) or other suitable chromatographic techniques. Thus, in some embodiments, the mass spectrometric technique is directly or indirectly coupled with a liquid chromatography technique, such as column chromatography, fast protein liquid chromatography (FPLC), ion exchange chromatography, size exclusion chromatography, affinity chromatography, high performance liquid chromatography (HPLC), nano-reverse phase liquid chromatography (nano-RPLC), poly acrylamide gel electrophoresis (PAGE), capillary electrophoresis (CE) or reverse phase high performance liquid chromatography (RP-HPLC) to further resolve the biological sample prior to mass spectrometric analysis.

The regents (such as buffers and the like) used in accordance with the disclosed methods are preferable chosen such as to not significantly interfere with mass spectral analysis, such as tandem mass spectrometric methods. Preferably, but not necessarily, the reagents are selected so as to impart desirable characteristics to the analysis. Examples of such characteristics include for example decreasing the energy required to volatilize the lipids, facilitating ionization, creating predominantly singly charged ions, reducing the peak width, and increasing the sensitivity and/or selectivity of the desired analysis product.

In embodiments, detecting the composition of lipids present in the extracted sample includes mass spectral analysis, chromatography or a combination thereof. In embodiments, detecting the composition of lipids present in the extracted sample includes LC-MS/MS using an untargeted lipidomic approach. In embodiments, ultra performance Liquid Chromotography Time-of-flight (UPLC-TOF) is utilized for trace level quantification with high level of sensitivity.

Following the determination of the lipid profile, the results, findings, diagnoses, predictions and/or treatment recommendations can be provided to the subject. For example, the results, findings, diagnoses, predictions and/or treatment recommendations can be recorded and communicated to technicians, physicians and/or patients, pharmacies, or clients. In certain embodiments, computers can be used to communicate such information to interested parties, such as, clients, patients and/or the attending physicians. Based on the measurement, the therapy or protocol administered to a subject can be started, modified not started or re-started. In some examples, the output can provide a recommended therapeutic regimen or skin care protocol. In some examples, the test may include determination of other clinical information.

In several embodiments, identification of a subject as having or at risk of developing a skin condition or disorder, such as AD, eczema, and or bacterial infection, results in the physician treating the subject, such as prescribing one or more therapeutic agents for inhibiting or delaying one or more signs and symptoms associated with the disorder/condition. In additional embodiments, the treatment, dose or dosing regimen is modified based on the information obtained using the methods disclosed herein.

The subject can be monitored while undergoing treatment using the methods described herein in order to assess the efficacy of the treatment protocol. In this manner, the length of time or the amount given to the subject can be modified based on the results obtained using the methods disclosed herein. The subject can also be monitored after the treatment using the methods described herein to monitor for relapse and thus, the effectiveness of the given treatment. In this manner, whether to resume treatment can be decided based on the results obtained using the methods disclosed herein. In some examples, this monitoring is performed by a clinical healthcare provider. Transepidermal water loss, Serum IgE, eosinophis and/or TARO levels can be determined as an indication of the mitigation of the disease progression and AD-pathogenesis.

In some embodiments, once a subject's lipid profile is determined, an indication of that profile can be displayed and/or conveyed to a clinician or other caregiver. For example, the results of the test are provided to a user (such as a clinician or other health care worker, laboratory personnel, or patient) in a perceivable output that provides information about the results of the test. In some examples, the output is a paper output (for example, a written or printed output), a display on a screen, a graphical output (for example, a graph, chart, or other diagram), or an audible output.

In other examples, the output is a numerical value, such as an amount of a particular set of lipids in the lipid profile as compared to a control. In additional examples, the output is a graphical representation, for example, a graph that indicates the value (such as amount or relative amount) of the set of lipids in the sample from the subject on a standard curve. In a particular example, the output (such as a graphical output) shows or provides a cut-off value or level that indicates the presence of optimal, sub-optimal or deficient lipid level. In some examples, the output is communicated to the user, for example by providing an output via physical, audible, or electronic means (for example by mail, telephone, facsimile transmission, email, or communication to an electronic medical record).

The output can provide quantitative information (for example, an amount of a lipid in a test sample compared to a control sample or value) or can provide qualitative information (for example, a diagnosis of a deficiency in a class or classification of lipids). In additional examples, the output can provide qualitative information regarding the relative amount of a particular lipid in the sample, such as identifying presence of an increase relative to a control, a decrease relative to a control, or no change relative to a control.

In some examples, the output is accompanied by guidelines for interpreting the data, for example, numerical or other limits that indicate the presence or absence of a disorder/condition. The indicia in the output can, for example, include normal or abnormal ranges or a cutoff, which the recipient of the output may then use to interpret the results, for example, to arrive at a diagnosis, prognosis, susceptibility towards or treatment plan.

### Compositions and Method of Treatment

Currently the skin creams and lotions in the market are a generic, “one-for-all” mixture of petrolatum and lipids with no specific ingredients that would meet the requirements of a specific individual. To meet this need, disclosed herein is a method of using the unique lipid profile of a subject to design a topical supplementation of lipids in the form of cream or lotion useful in restoring normal skin or lipid barrier and mitigating a disease phenotype. In some embodiments, the method further includes providing an appropriate therapy and/or protocol for the subject diagnosed with a skin lipid deficiency, for example administering or providing a topical supplementation of lipids in the form of cream or lotion to ameliorate a skin lipid deficiency. In some examples, a subject is selected that has, or is believed to have a skin condition, for example atopic dermatitis, eczema, psoriasis, ichthyosis Netherton Syndrome or any other skin diseases manifested with barrier disruption and monitored by increased trans epidermal water loss (TEWL).

Lipids can be obtained from any source, for example commercially available lipids can be obtained from Avanti Polar Lipids, Inc. Alabaster, Ala., and Matreya LLC, State College, Pa., amongst others.

Disclosed is a method of augmenting and/or treating a lipid deficiency in skin of a subject. In embodiments, the method includes, identifying a deficiency in one or more lipids in a skin sample obtained from the subject, such as by any of the forgoing methods disclosed herein. Once identified, a topical therapeutic composition is formulated (based on the identified lipid deficiency) that contains the one or more lipids, a similar lipid, or a subset thereof, found to be deficient. An effective amount of the formulation and/or composition is then provided and/or administered to the subject. In some embodiments, the method is a method of treating or inhibiting Staphylococcus aureus infection in the skin of the subject. An effective amount of agent that is sufficient to generate a desired response, such as reducing or inhibiting one or more signs or symptoms associated with a condition or disease. When administered to a subject, a dosage will generally be used that will achieve target tissue concentrations. In some examples, an “effective amount” is one that treats one or more symptoms and/or underlying causes of any of a disorder or disease. In some examples, the composition is one or more of the compositions disclosed below. It is contemplated that compositions with similar properties could be administered as well.

It is contemplated that the desired treatments or protocols may be administered via any means known to one of skill in the art, although topical administration is generally preferred. Any skin surface can be treated using the methods provided herein. By “skin surface” is intended the stratum corneum, epidermis, dermis or any other layer of the skin thereof. Skin surfaces that can be treated include, but are not limited to face, scalp, neck, chest, back, torso, arms, legs, hands or feet including periorbits, lips, cheeks, nasolabial folds, forehead, chin, neck, upper lip rhytides, or any combination thereof. The skin of any facial surface can be treated using the methods provided herein. The method can be applied to any facial or scalp area and/or to any body surface area, with other immediate areas of application being the chest, neck and body. More than one skin surface can be treated during the same treatment period.

The treatment can be performed multiple times for optimal results. In one embodiment, the treatment is performed twice a day. In another embodiment, the treatment is performed daily. In other embodiments, the treatment is performed weekly. In another embodiment, the treatment is performed monthly. In another embodiment, the treatment is performed at least once every one to two days. In another embodiment, the treatment is performed at least once every one to two weeks. In other embodiments, the treatment is performed as described below with one or more of the disclosed compositions.

As touched on above with respect to the assignment of skin deficiency category, once a skin deficiency category has been assigned, the subject can be provided with a formulation that includes lipids that are selected to ameliorate the lipid imbalance, for example ameliorate a lipid deficiency. Thus, in some embodiments, the subject is provided and/or administered a composition, such as a cream, that is formulated to ameliorate a lipid deficiency found in the subject's lipid profile. In some embodiments, and in particular when the subject has been to a skin lipid deficiency category, the method further includes providing a therapeutic composition formulated to ameliorate the lipid deficiency present in the skin lipid deficiency category. In embodiments (for group I), the composition comprises FFA 16:1 and FFA 18:1 which may be supplemented with the emollient or cream base containing oil/water emulsion. In embodiments comprises (for group II), the composition FFA 16:1, FFA 18:1, CER[AH]C48, CER[EOH]C66 and CER[EOH] C68 which may be supplemented with the emollient or cream base containing oil/water emulsion. In embodiments (for group III), the composition comprises FFA 16:1, FFA 18:1, CER[AP]C40, CER[NDS]C52, CER[NDS]C54, CER[AH]C48, CER[EOH]C66, CER[EOH] C68, and CER[EOS]C70 which may be supplemented with the emollient or cream base containing oil/water emulsion.

As disclosed herein, some lipid deficiencies have been identified as correlating to bacterial infection, or the susceptibility to bacterial infection. A subject with such a lipid deficiency would benefit from augmentation of their lipids with formulations that included those lipids identified as deficient, a similar lipid or a subset thereof. In some embodiments, a formulation includes lipids identified as involved in microbial defense, for example a formulation including one or more of the lipids set forth in Table 2A, such as one or more of FFA16:1, FFA18:1, TG48:1, TG48:2, TG50:1, TG50:2, TG50:3, TG58:2, CER[AH]C38, or CER[AP]C40. In some embodiments, a formulation includes lipid identified as involved in skin permeability barrier protection, for example a formulation including one or more of the lipids set forth in Table 2B, such as one or more of CER[NDS]C52, or CER[NDS]C54. In some embodiments, a formulation includes lipid identified as involved in anti-microbial defense and skin barrier protection, for example a formulation including one or more of the lipids set forth in Table 2C, such as one or more of TG46:2, CER[AH]C48, CER[EOH]C66, CER[EOH]C68, or CER[EOS]C70. In some embodiments, a formulation includes one or more of the lipids set forth in Table 3, such as one or more of CER[AH]C38, CER[AH]C48, CER[AP]C40, CER[NDS]C52, CER[NDS]C54, CER[EOH]C66, CER[EOH]C68, CER[EOS]C70, FFA16:1, FFA18:1, TG46:2, TG48:1, TG48:2, TG50:1, TG50:2, TG50:3, or TG58:2.

Also disclosed is a method of treating a skin ailment that includes providing or administering a personalized formulation to ameliorate a lipid deficiency, for example if the composition provided for the assigned category is not sufficient or does not provide the desired outcome. In such a situation, a personalized topical medicament is prepared for treating the lipid imbalance and provided and/or administered to the subject. A disclosed composition includes one or more lipids identified as deficient, such as 2 or more, 3 or more, 4 or more, 5 or more, 6 or more, 7 or more, 8 or more, 9 or more, 10 or more, 12 or more, 15 or more, 10 or more, 50 or more, 100 or more or a greater number of lipids within a single class or within 2 or more, 3 or more, 4 or more, 5 or more, 6 or more, 7 or more or a greater number of different lipid classes, which may include any of the afore mentioned lipids.

In embodiments, the present disclosure provides methodology to develop cream, lotion, and emollients that are tailored to meet the unique needs of an individual as determined by characterization of the individual's skin lipid profile. In embodiments, the formula would be 2 or more, 3 or more, 4 or more, 5 or more, 6 or more, 7 or more, 8 or more, 9 or more, 10 or more, 12 or more, 15 or more, 10 or more, 50 or more, 100 or more or a greater number of lipids within a single class or within 2 or more, 3 or more, 4 or more, 5 or more, 6 or more, 7 or more or a greater number of different lipid classes, which may include any of the afore mentioned lipids.

The lipid formulations disclosed herein can be applied in within a cream or base. Such formulations can include a cold cream base, also referred to a without Emulsion Base (Cold Cream type base). There are five (5) classes or types of ointment bases which are differentiated on the basis of their physical composition. These are: oleaginous bases; absorption bases; water in oil emulsion bases; oil in water emulsion bases; and water soluble or water miscible bases. An exemplary formulations are shown below. Each ointment base type has different physical characteristics and therapeutic uses based upon the nature of its components. The pharmaceutically acceptable carriers (vehicles) useful in formulations are conventional and can be found in The Science and Practice of Pharmacy, Loyd V. Allen, Jr, editor. Philadelphia, Pa.: Pharmaceutical Press (2012).

BASE NO. I: Oleaginous Base (White Ointment)

| | |
|---|---|
| White Wax | 5% |
| White Petrolatum | 95% |

Procedure for Preparation:
a. Melt the white wax on a hot plate. No need to heat beyond 70-75° C.
b. When the wax has completely melted, add the petrolatum and allow the entire mixture to remain on the hot plate until liquefied.
c. Following liquefication, remove from heat and allow the mixture to congeal. Stir the mixture until it begins to congeal.

BASE NO. II: Absorption Base

| | |
|---|---|
| Cholesterol | 3% |
| Stearyl Alcohol | 3% |
| White Wax | 8% |
| White Petrolatum | 86% |

Procedure for Preparation:
a. Melt the stearyl alcohol, white wax, and petrolatum together on a hot plate.
b. Add the cholesterol to the mixture; stir until completely dissolved.
c. Remove the mixture from the hot plate and stir until congealed.

BASE NO. III: W/O Emulsion Base (Cold Cream type base)

| | |
|---|---|
| White wax | 12.0% |
| Cetyl Esters Wax (or Spermaceti) | 12.5% |
| Mineral Oil (Sp Gr = 0.9) | 56.0% |
| Sodium Borate | 0.5% |
| Water | 19.0% |

Procedure for Preparation:
a. Melt the white wax and spermaceti on a hot plate.
b. Add the mineral oil to this mixture and bring the temperature to 70° C.
c. Dissolve the sodium borate in water.
d. Heat the sodium borate solution to 70° C.
e. When both phases have reached the desired temperature, remove both phases from the hot plate and add the aqueous phase slowly and with constant stirring to the oil phase.
f. Stir briskly and continuously until congealed.

BASE NO. IV: O/W Emulsion Base (Hydrophilic Ointment)

| | |
|---|---|
| Sodium Lauryl Sulfate | 1.0% |
| Propylene Glycol (SP Gr = 1.035) | 12.0% |
| Stearyl Alcohol | 25.0% |
| White Petrolatum | 25.0% |
| Purified Water | 37.0% |

Procedure for Preparation:
a. Melt the stearyl alcohol and white petrolatum on a hot plate.
b. Heat this mixture to 70° C.
c. Dissolve remaining ingredients in water and heat the solution to 70° C.
d. Add the oleaginous phase slowly to the aqueous phase, stirring constantly.
e. Remove from heat and stir the mixture until it congeals.

BASE NO. V: Water Soluble Base

| | |
|---|---|
| Polyethylene Glycol 400 | 60% |
| (SP Gr = 1.12) | |
| Polyethylene Glycol 3350 | 40% |

Procedure for Preparation:
a. Melt the PEG 400 and Carbowax 3350 on a hot plate.
b. Warm the mixture to about 65° C.
c. Remove from the hot plate and stir until congealed.

The following examples are provided to illustrate certain particular features and/or embodiments. These examples should not be construed to limit the invention to the particular features or embodiments described.

### EXAMPLES

### Example 1

### Lipid Extraction

This example describes an exemplary method for the extraction of lipids from skin tape strips.

1) Take 4th-8th tapes per subject, for example as collected using a D-SQUAME standard skin sampling discs (available on the world wide web at store.cuderm.com) and the a D-SQUAME Pressure Instrument D500.

2) Add extraction solvent: CHCl₃:CH₃OH:H₂O (1:2:0.5), 1 milliliter, incubation time 1 hour at RT, vortex 1 minutes. While other extraction solvents can be used, this extraction solvent is optimal for the purpose of getting maximum yield of lipid from corneocytes of the skin the mixture.

3) Add 3 microliters of internal standard to each tube. Samples can be incubated for a period of time, for example 1 hour to facilitate phase separation;

4) Centrifuge at 2,000 r.p.m. for 10 minutes.

5) Transfer the chloroform phase to new glass vial and lipid obtained from 3 tapes (each individual will be pooled).

6) Dry under nitrogen (LPSC) (Can be stored in −80° C.).

7) Reconstitute the lipid in 100 μl methylene chloride:isopropanol:methanol=25:10:65 and briefly centrifuge.

8) Take the supernatant and use for LC/MS.

The above lipid extraction method provides a fast, simple, and reliable one-step method for isolation of skin lipids from a limited number corneocytes retrieved by non-invasive tape stripping method. After extraction of the lipids, lipidomic analysis is performed using, for example, mass spectrometry. Lipidomic analysis is used to characterize a subject's lipid profile, i.e., the determine levels of specific subgroups of ceramides, e.g., saturated and unsaturated, cholesterol and free fatty acids.

### Example 2

### Example of Personalized Formulations

Personalized formulations are formulated that include at least one lipid similar to a skin lipid or lipids that are deficient, in excess, or otherwise abnormal in the lipid profile of a subject. For example, based on the free fatty acids chain length, saturated and unsaturated ceramides may be short (less than 8 carbons), medium (8 to 14 carbons) or long (14 or more carbons) ceramides. In another example, a personalized formulation may include at least one free fatty acid, cholesterol, saturated ceramide, or unsaturated ceramide that is the same or similar to a skin lipid that is deficient, in excess, or otherwise abnormal in the lipid profile of a subject.

In yet another example, a personalized formulation is any lipid based composition that, when applied to the skin of a subject, improves or restores the barrier function of the skin, such that symptoms of a skin disorder are alleviated or eliminated.

### Example 3

### Altered Composition of Epidermal Lipids Correlates Atopic Dermatitis

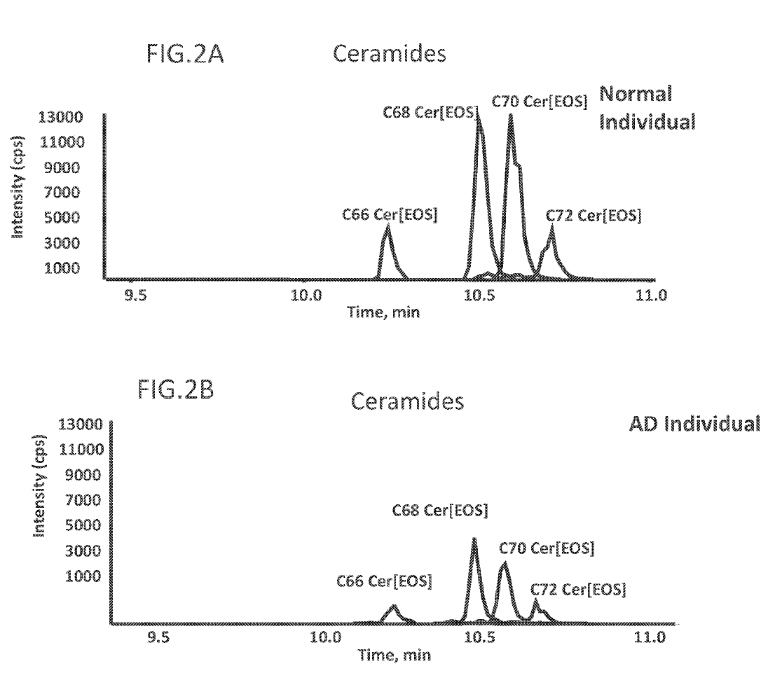

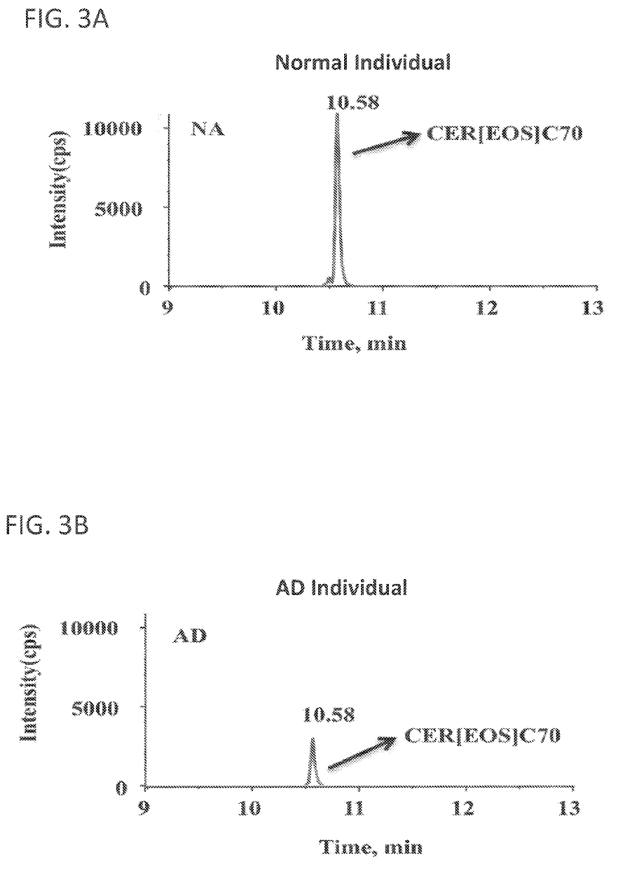

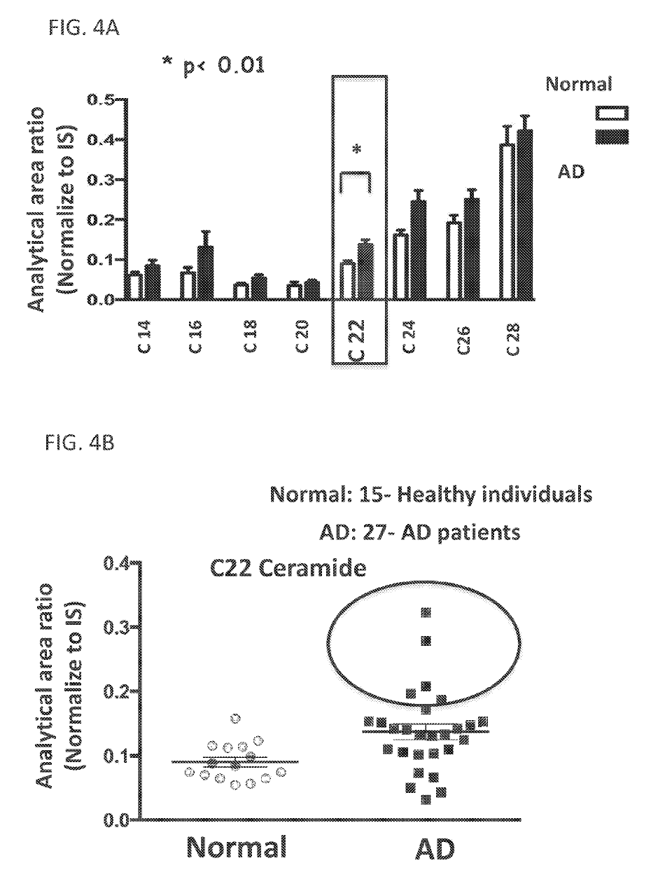

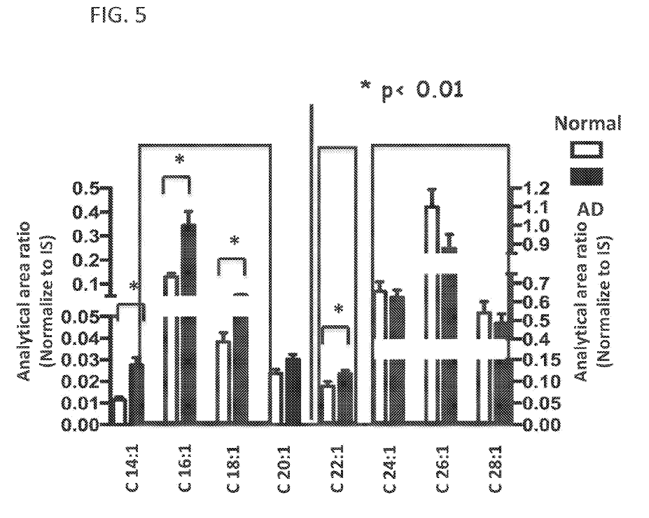

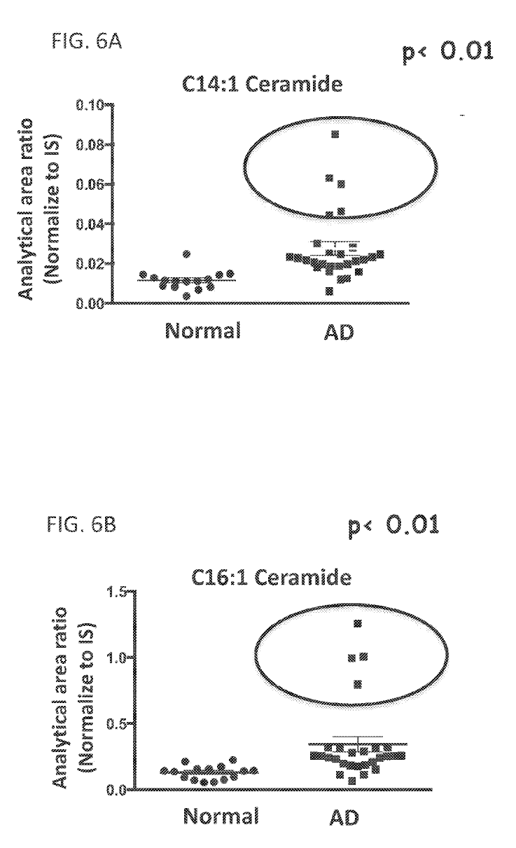

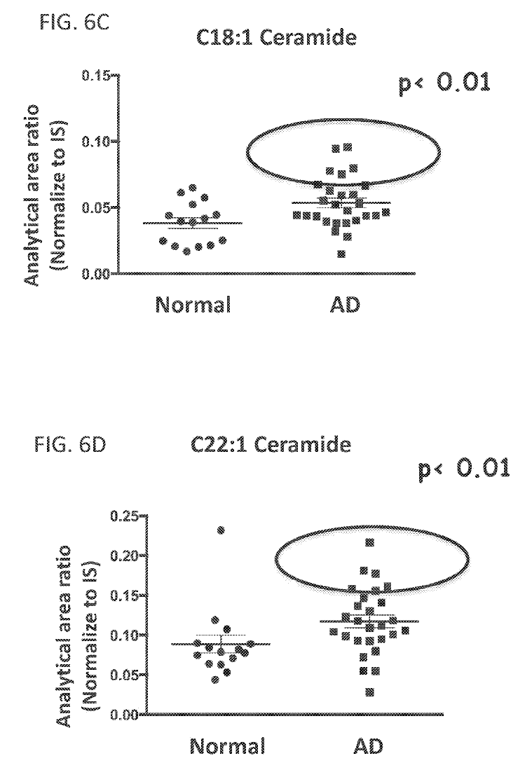

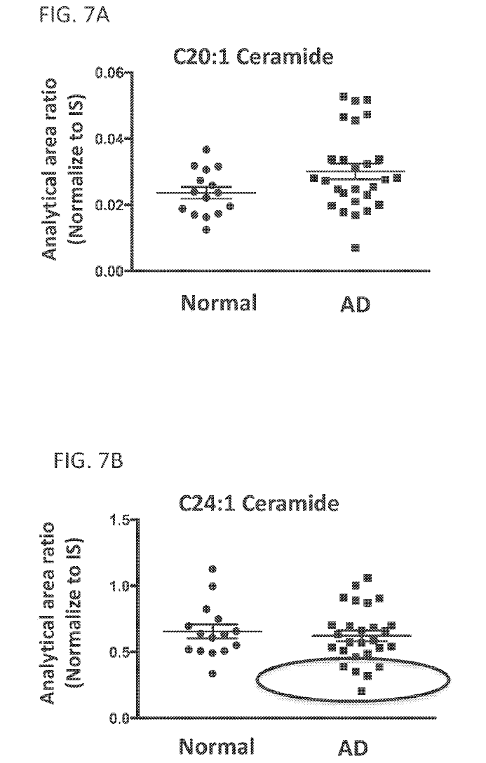

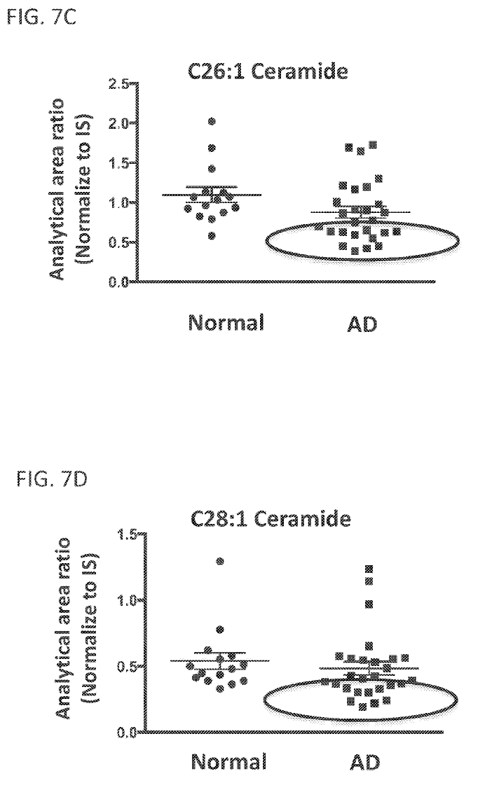

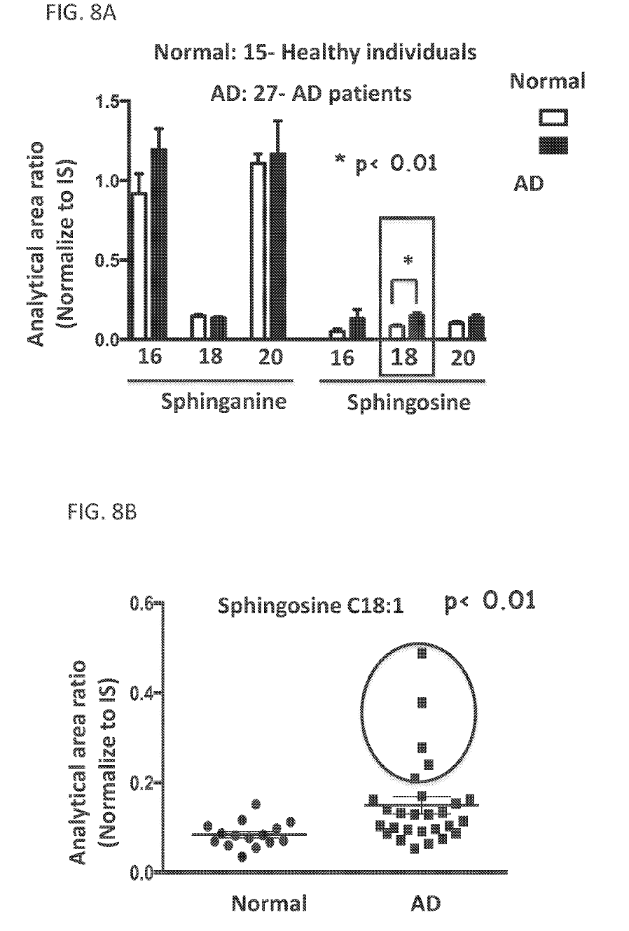

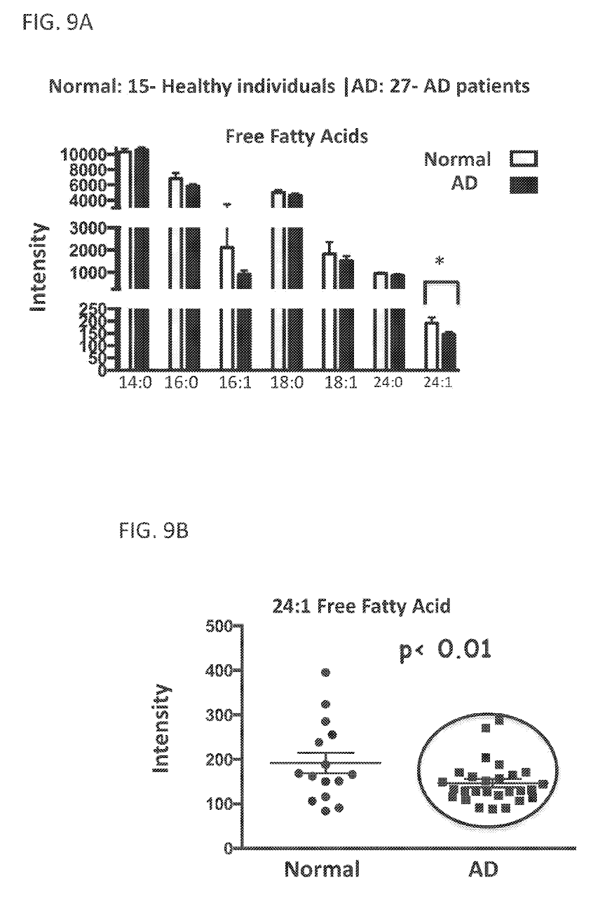

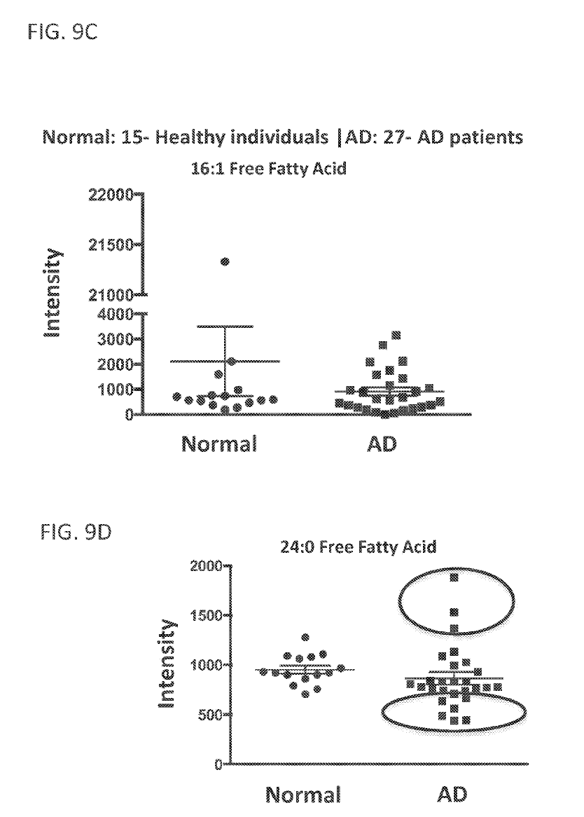

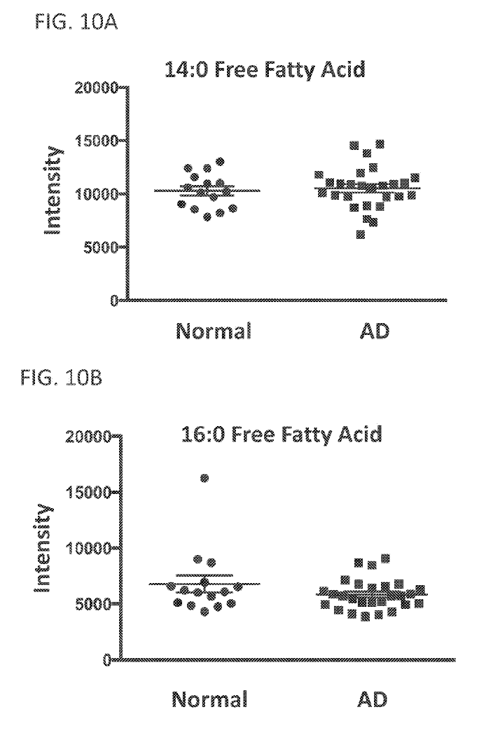

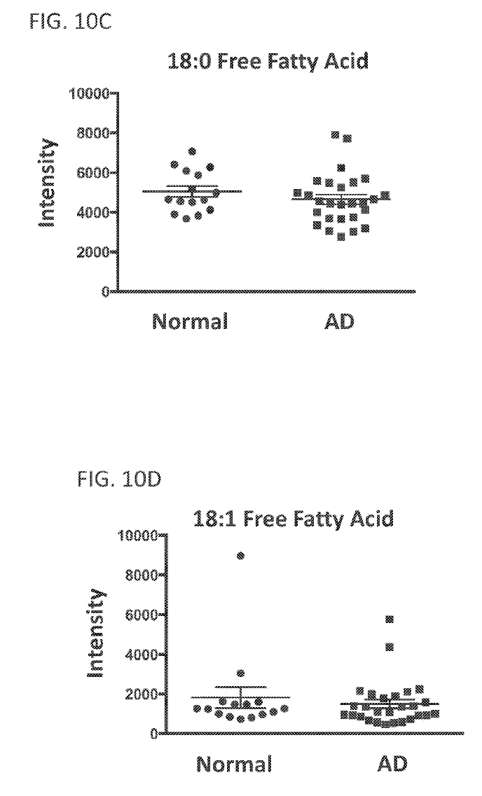

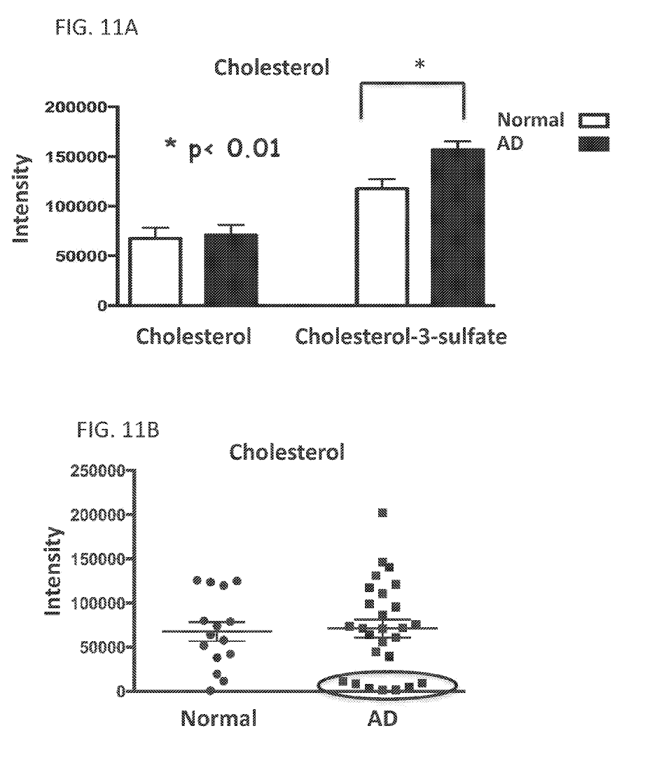

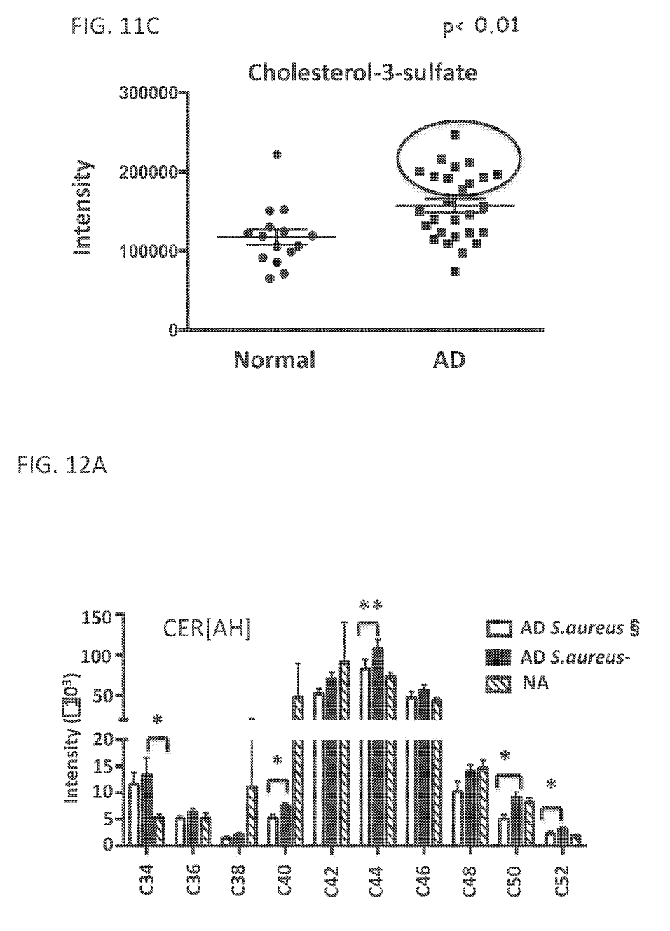

The present disclosure demonstrates, as shown in FIGS. 2-11, that specific subgroups of lipids, including ceramides (see FIGS. 3-8) (saturated and unsaturated), cholesterol (see FIG. 11), and free fatty acids (see FIGS. 9 and 10), were either reduced or in disproportion in AD-individuals whose skin surface lipids were measured. Accordingly, a topical supplementation of lipids in the form of cream or lotion on the skin of the affected individuals (cats, dogs, and humans) would be very useful to restore a more normal barrier, decrease onset of the disease, mitigate the disease phenotype, and significantly improve the quality of life. As is shown in FIGS. 2-8, disease characterization through lipidomics analyses illustrates an altered lipid composition in human atopic dermatitis patients with defective barrier function. This group of patients had an an enhanced saturated (C22) and un-saturated (C 14:1, C16:1, C18:1 and C22:1) ceramides and saturated sphingosine (C18) in specific subgroup of AD-individuals, a reduced un-saturated ceramides (C24:1, C26:1 and C28:1) in specific AD-subgroup. Disease characterization through lipidomics analyses further identified a specific subgroup of AD-individuals with reduced free fatty acids (FFA) [C24 and C24:1] (see FIGS. 9 and 10) and cholesterol (see FIGS. 11A-11B), and an enhanced cholesterol sulfate in a selective subgroup of AD-individuals (see FIG. 11C) Table 1 lists identified lipids that are significantly reduced in the atopic dermatitis subjects compared to normal healthy individuals independent of Staph status. The ranges in healthy individuals are indicated

TABLE 1
List of identified lipids that are significantly reduced in the atopic
dermatitis subjects compared to normal healthy individuals

| Lipids | Mean ± SD |
|---|---|
| CER[AH]C48 | 14562 ± 6261 |
| CER[AP]C40 | 11644 ± 4132 |
| CER[NDS]C52 | 20611 ± 6908 |
| CER[NDS]C54 | 5843 ± 2317 |
| CER[EOH]C66 | 13234 ± 3966 |
| CER[EOH]C68 | 16398 ± 5247 |
| CER[EOS]C70 | 12281 ± 4829 |
| FFA16:1 | 743 ± 522 |
| FFA18:1 | 1315 ± 575 |

One subject with extremely high level of FFA16:1 and FFA18:1, otherwise, the Mean±SD will be 2114±5339, and 1826±2052. The SC lipids were extracted from 15 healthy individuals and subjected to LC-MS/MS analysis. Data was imported into the software (e.g PeakView) for relative quantification and identification. The intensity (counts per second) of lipids was normalized to specific internal standards. The normal lipid range was reported in Mean±SD.

Table 2 shows the correlation between lipid composition of clinical AD subphenotypes of S. aureus colonized subjects and TEWL. All altered (reduced) lipid sub-groups are listed based on predicted functions.

TABLE 2
Correlation between lipid composition and clinical AD
subphenotypes of S. aureus colonized or barrier
disrupted (Lipid sub-groups based on function)

| | | | |
|---|---|---|---|
| | NA staph− vs | AD staph− vs | |
| | AD staph− | AD staph+ | Basal TEWL |
| Lipids | p-value | p-value | p-value |

A. Lipids involved in anti-microbial defense

| | | | |
|---|---|---|---|
| FFA16:1 | 0.2884 | <0.01 | 0.7818 |
| FFA18:1 | 0.0605 | <0.001 | 0.5345 |
| TG48:1 | 0.1805 | <0.01 | 0.2727 |
| TG48:2 | 0.2996 | <0.01 | 0.1343 |
| TG50:1 | 0.0602 | <0.05 | 0.4717 |
| TG50:2 | 0.0631 | <0.01 | 0.4298 |
| TG50:3 | 0.0643 | <0.01 | 0.3807 |
| TG58:2 | 0.4289 | <0.01 | 0.7223 |
| CER[AH]C38 | 0.0514 | <0.05 | 0.6599 |
| CER[AP]C40 | 0.4511 | <0.05 | 0.2608 |

B. Lipids involved in skin permeability barrier protection

| | | | |
|---|---|---|---|
| CER[NDS]C52 | <0.05 | 0.8270 | <0.01 |
| CER[NDS]C54 | <0.05 | 0.8931 | <0.01 |

C. Lipids involved in anti-microbial defense and
skin barrier protection

| | | | |
|---|---|---|---|
| TG46:2 | 0.5321 | <0.05 | <0.05 |
| CER[AH]C48 | 0.8534 | <0.05 | <0.001 |
| CER[EOH]C66 | 0.7973 | <0.01 | <0.001 |
| CER[EOH]C68 | 0.5206 | <0.01 | <=0.0010 |
| CER[EOS]C70 | 0.1251 | <0.05 | <0.01 |

Abbreviations:
NA, non-atopic healthy individuals;
AD, atopic dermatitis,
staph+, Staph aureus positive,
staph−, Staph aureus negative,
TEWL, Trans-epidermal water loss (an indicator/marker of AD phenotype)
All represented data are after age and gender adjustments

### Example 4

### Altered Composition of Epidermal Lipids Correlates with Staphylococcus aureus Colonization Status in Atopic Dermatitis Subjects

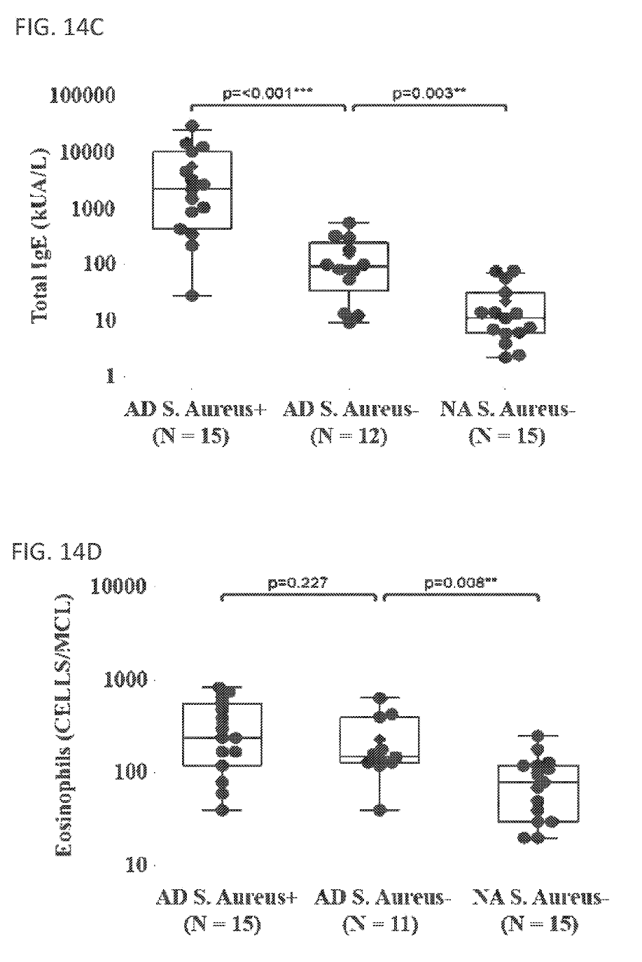

Transepidermal water loss (TEWL), serum thymus and activation-regulated chemokine (TARC/CCL17), IgE and eosinophil counts are useful clinical markers in diagnosis and assessment of AD. Analyses of all of the above markers, revealed an elevated TEWL, serum TARC, IgE levels and eosinophil counts in all AD patients compared to healthy individuals (FIGS. 14A-14D). SC lipids were extracted by a high-yield, one-step, method and analyzed by modified LC/MS/MS. The profile of all major SC lipids including CERs, FFAs, Cholesterol and TGs was compared between AD-S. Aureus+, AD-S. Aureus− and non-atopic (NA) subjects (see methods below).

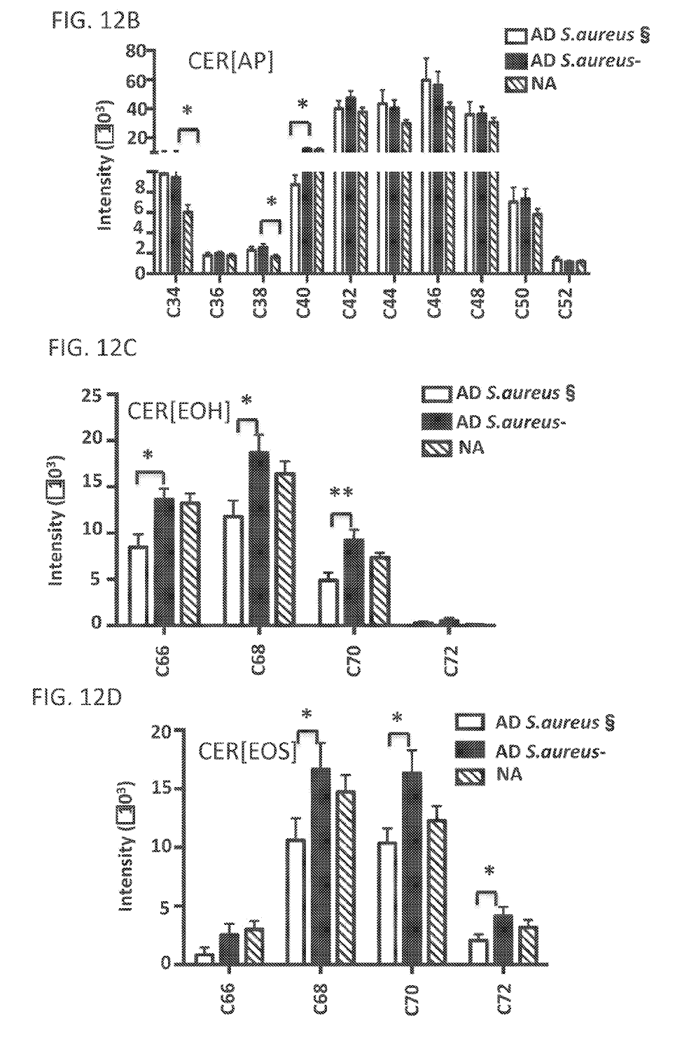

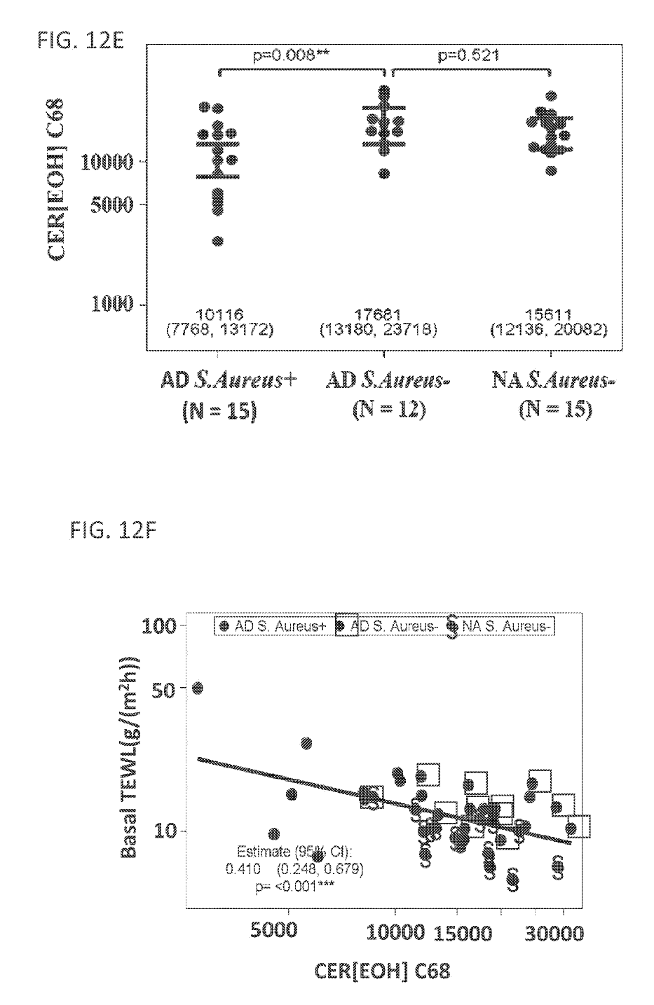

CERs are the most abundant lipid class in human SC (50%) and divided into 12 subclasses (van Smeden J, Janssens M, Gooris G S, Bouwstra J A. The important role of stratum corneum lipids for the cutaneous barrier function. Biochim Biophys Acta 2014; 1841:295-313). Altered CERs composition and organization in AD-patients with skin barrier dysfunction has been noted (van Smeden J, Janssens M, Gooris G S, Bouwstra J A. The important role of stratum corneum lipids for the cutaneous barrier function. Biochim Biophys Acta 2014; 1841:295-313). First, the CER profiles of all AD patients were compared to those of NA subjects. The data showed that certain short-chain CERs, such as CER[AH]C34 and CER[AP]C34, were significantly higher in AD subjects, which was consistent with previous report that increased in short-chain ceramides correlates with decreased barrier function (van Smeden J, Janssens M, Gooris G S, Bouwstra J A. The important role of stratum corneum lipids for the cutaneous barrier function. Biochim Biophys Acta 2014; 1841:295-313). Next, the CER profiles of AD patients were compared based on S. aureus colonization status. Specific CERs, belonging to 4 out of 12 CERs subclasses, were altered in AD-S. Aureus+ compared to AD-S. Aureus− subjects. The levels of CER[AH] (40 and 50 carbons length), CER[AP] (40 carbons length), as well as most detectable long-chain CERs, such as CER[EOH](e.g 66, 68 and 70 carbons length) and CER[EOS](e.g 68,70 and 72 carbons lengths), were significantly lower in skin of AD-S. Aureus+ in comparison to AD-S. Aureus− individuals (FIGS. 12A-D). After age and gender adjustment in each group, CER[AH]C38, CER[AH]C48, CER[AP]C40, CER[EOH]C66, CER[EOH]C68 and CER[EOS]C70 were identified to be significantly lower in AD patients based on S. aureus colonization (TABLE 3). Furthermore, an association between lipidomics data and measures of barrier integrity was estimated. Diminished levels of certain CERs including CER[NDS]C52 and CER[NDS]C54 (TABLE 3) in skin SC negatively correlated to increased TEWL. The relationship between CERs levels and TEWL values indicated that this subgroup of lipids might be involved in epidermal permeability barrier (EPB) homeostasis. In another subgroup, CERs, such as CER[AP]C40 (TABLE 3), were significantly lower in AD-S. Aureus+ compared with AD-S. Aureus− subjects, and comparable between AD-S. Aureus− and NA subjects. However, that decrease did not correlate with TEWL values, indicating that those CERs might exhibit antimicrobial activities. Level of CER[EOH]C68, the most abundant long-chain CERs in skin SC, was significantly decreased in AD-S. aureus+ subjects (10116vs. 17681, p=0.008) and negatively correlated to TEWL (FIGS. 12E and 12F). This is in agreement with a previous report that a reduction in long-chain CERs in AD patients leads to an abnormal lipid organization, thereby resulting in disrupted barrier functions (van Smeden J, Janssens M, Gooris G S, Bouwstra J A. The important role of stratum corneum lipids for the cutaneous barrier function. Biochim Biophys Acta 2014; 1841:295-313).

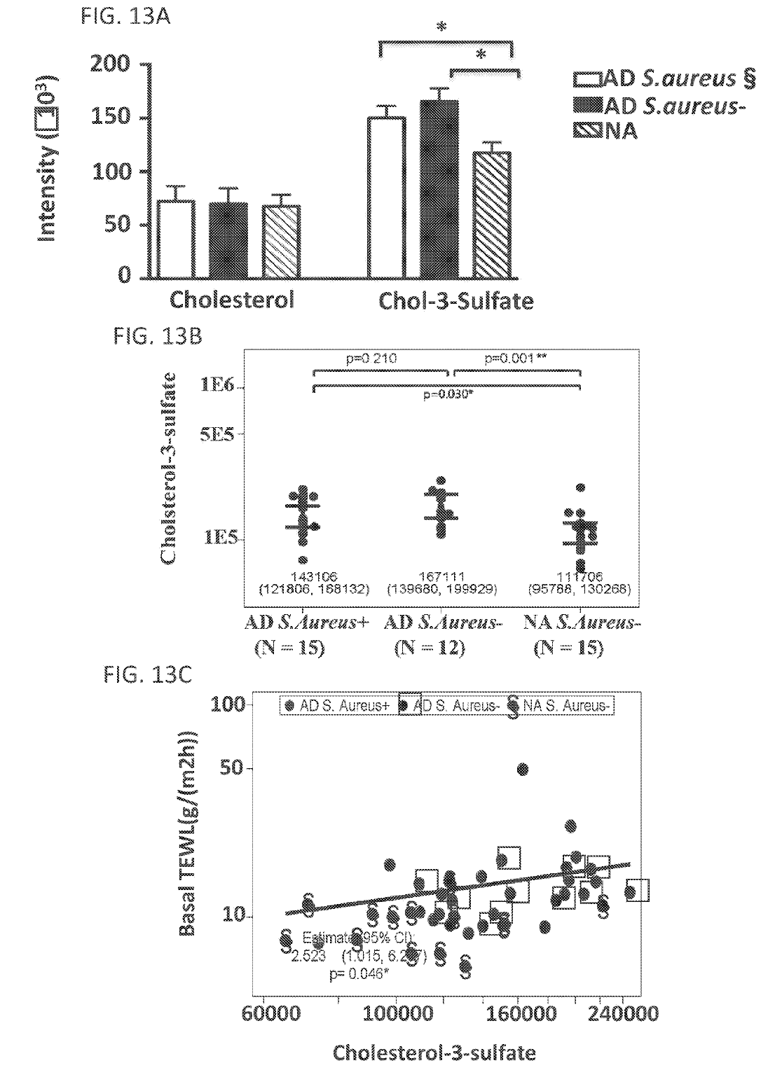

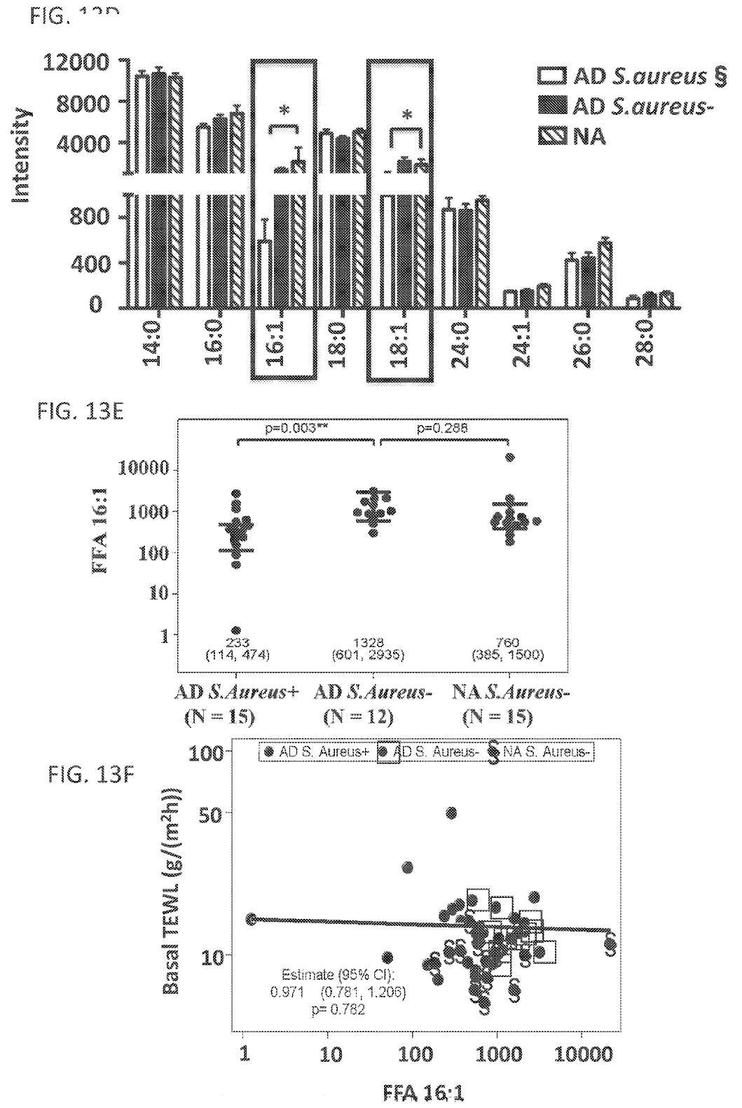

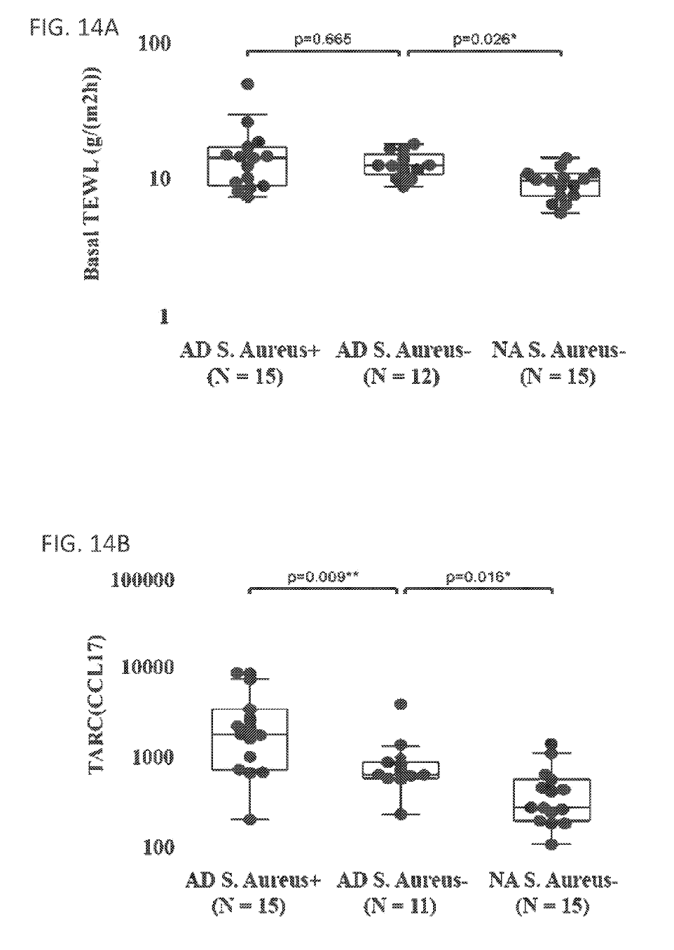

Cholesterol and cholesterol-sulfate are abundant in human epidermis. In the studies disclosed herein of an AD population, the level of cholesterol was comparable between AD and NA subjects, and unchanged even after S. aureus-subphenotype (FIGS. 14A and B). It has been recently reported that cholesterol level was comparable between AD and normal individuals (Joo K M, Hwang J H, Bae S, Nahm D H, Park H S, Ye Y M, et al. Relationship of ceramide-, and free fatty acid-cholesterol ratios in the stratum corneum with skin barrier function of normal, atopic dermatitis lesional and non-lesional skins. J Dermatol Sci 2015; 77:71-4). Interestingly, an increase in cholesterol-3-sulfate was observed in both AD-S. Aureus− and AD-S. Aureus+ subjects compared to NA subjects, irrespective of age and gender adjustment, and positively correlated to increased TEWL (FIGS. 13A-C), suggesting its role in altered EPB homeostasis. A recent study also indicated that disruption of cholesterol-sulfate cycle accounts for EPB abnormality in X-linked ichthyosis (Elias P M, Williams M L, Choi E H, Feingold K R. Role of cholesterol sulfate in epidermal structure and function: lessons from X-linked ichthyosis. Biochim Biophys Acta 2014; 1841:353-61).

Fatty acids, a major constituents of the SC, are crucial for barrier functions (Feingold K R, Elias P M. Role of lipids in the formation and maintenance of the cutaneous permeability barrier. Biochim Biophys Acta 2014; 1841:280-94). FFAs chain length was reported to be altered in AD skin (van Smeden J, Janssens M, Gooris G S, Bouwstra J A. The important role of stratum corneum lipids for the cutaneous barrier function. Biochim Biophys Acta 2014; 1841:295-313). It was observed that the level of the very long chain FFA24:1 and FFA26:0 were lower in AD-subjects compared to NA. After further S. aureus-subphenotyping, levels of two unsaturated FFAs, FFA16:1 and FFA18:1, were significantly lower in AD-S. Aureus+ compared to those of AD-S. Aureus− (FIGS. 13D and 13E), and comparable between AD-S. Aureus− and NA subjects. Alteration of these FFAs did not correlate with increased TEWL (FIG. 13F), suggesting a possible involvement in skin antimicrobial defense. The antimicrobial activities of FFA16:1 and FFA18:1 against S. aureus in vitro was further evaluated and it was observed that FFA 16:1 exhibited potent antibacterial activity. This observation is consistent with previous findings that toxic exogenous FFA16:1 was a potent bacterial growth inhibitor, while FFA18:1 was nontoxic (Parsons J B, Yao J, Frank M W, Jackson P, Rock C O. Membrane disruption by antimicrobial fatty acids releases low-molecular-weight proteins from Staphylococcus aureus. J Bacteriol 2012; 194:5294-304).

TGs that are synthesized in keratinocytes and normally broken down to FFAs, play a critical role in EPB maintenance (Feingold K R, Elias P M. Role of lipids in the formation and maintenance of the cutaneous permeability barrier. Biochim Biophys Acta 2014; 1841:280-94; Radner F P, Fischer J. The important role of epidermal triacylglycerol metabolism for maintenance of the skin permeability barrier function. Biochim Biophys Acta 2014; 1841:409-15). In order to determine whether TGs are associated with susceptibility to S. aureus in AD, the TGs profile was examinered in AD subjects. Notably, level of a group of TGs (e.g TG46:2, TG48:1, TG48:2, TG50:1, TG50:2, TG50:3, TG58:2) was significantly lower in AD-S. Aureus+compared to AD-S. Aureus− subjects after age and gender adjustments and only decrease in TG46:2 significantly correlated to altered TEWL (TABLE 3). Above results suggested possible involvement of TGs in other cellular processes including skin anti-microbial defense.

It has been recently reported that among 2430 differentially expressed genes in AD skin, those involved in lipid metabolism and biosynthesis were in the top list by GO-term analysis, indicating importance of lipid metabolism in atopic skin.¹¹ It remains to be identified if the present alteration of lipid composition in AD-S. Aureus+ subjects is due to abnormal expression of lipid metabolism genes. Altogether, characteristic lipid profile is a candidate marker for diagnosis and prediction of S. aureus susceptibility in AD. Therefore, measurement of skin lipid composition is a fast, reliable and non-invasive tool to characterize AD-subtypes and could be important for developing individualized cream with specific lipid species for S. Aureus+ or S. Aureus− AD patients.

TABLE 3
Correlation between lipid composition of clinical AD
subphenotypes of S. aureus colonized subjects and TEWL
(All altered/reduced lipids are listed after removal
of the subgroups)

| | NA staph− vs AD | AD staph− vs AD | |
|---|---|---|---|
| | staph− | staph+ | Basal TEWL |
| Lipids | p-value | p-value | p-value |
| CER[AH]C38 | 0.0514 | <0.05 | 0.6599 |
| CER[AH]C48 | 0.8534 | <0.05 | <0.001 |
| CER[AP]C40 | 0.4511 | <0.05 | 0.2608 |
| CER[NDS]C52 | <0.05 | 0.8270 | <0.01 |
| CER[NDS]C54 | <0.05 | 0.8931 | <0.01 |
| CER[EOH]C66 | 0.7973 | <0.01 | <0.001 |
| CER[EOH]C68 | 0.5206 | <0.01 | <=0.001 |
| CER[EOS]C70 | 0.1251 | <0.05 | <0.01 |
| FFA16:1 | 0.2884 | <0.01 | 0.7818 |
| FFA18:1 | 0.0605 | <0.001 | 0.5345 |
| TG46:2 | 0.5321 | <0.05 | <0.05 |
| TG48:1 | 0.1805 | <0.01 | 0.2727 |
| TG48:2 | 0.2996 | <0.01 | 0.1343 |
| TG50:1 | 0.0602 | <0.05 | 0.4717 |
| TG50:2 | 0.0631 | <0.01 | 0.4298 |
| TG50:3 | 0.0643 | <0.01 | 0.3807 |
| TG58:2 | 0.4289 | <0.01 | 0.7223 |

Abbreviations:
NA, non-atopic healthy individuals;
AD, atopic dermatitis,
staph+, Staph aureus positive,
staph−, Staph aureus negative,
TEWL, Trans-epidermal water loss (an indicator/marker of AD phenotype).
All represented data are after age and gender adjustments.

Methods

Study Subjects

27 subjects with AD and 15 healthy individuals with no history of skin disorders (>=18 years) were enrolled under IRB-approved protocol. 15 out of 27 AD subjects were subphenotyped as AD-S. aureus+ since the growth of S. aureus from skin swabs obtained at lesional or nonlesional sites. 12-remain AD subjects and all non-atopic subjects had no growth of S. aureus and were subphenotyped as AD-S. aureus− and NA subjects.

Sample Collection from the Human Skin

To obtain SC specimens, the D-SQUAME standard skin sampling discs with diameter 22.0 mm were pressed on the non-lesional (unaffected) skin of patients with AD or healthy individuals and stripped. The D-SQUAME pressure instrument D500 was used to apply all tape strips using 225 gcm-2 of pressure. A total of 20 consecutive discs were collected from each individual. The tapes were store at −80° C. until lipid extraction.

Lipid Extraction from the SC

Lipid was extracted using modified Bligh and Dyer method. Briefly, 4 consecutive tapes (#5th-#8th) per subjects were incubated in extraction solvent (chloroform:methanol:water 1:2:0.5) at room temperature for 1 hour. A volume of 2.5 ul of internal standard mixture (Avanti, Alabaster, Ala.) was added to 1 ml extraction solvent before incubation. After incubation, extraction solvents from each individual were pooled. After centrifugation at 2.000 rpm for 10 min, lower chloroform phase was collected and dried under nitrogen. The samples were reconstituted in methylene chloride:isopropanol:methanol at ratio of 25:10:65.

Ultrahigh-Pressure Liquid Chromatography/MS/MS (LC/MS/MS)

Ultra-pressure liquid chromatography was performed on a Shimadzu Nexera system (Shimadzu, Columbia, Md.) coupled with a quadrupole time-of-flight mass spectrometer (AB SCIEX, Triple TOF 5600) operated in information dependent MS/MS acquisition mode. The column (1.8 μm particle 100×2.1 mm id HSS T3 column (Waters, Milford, Mass.)) was heated to 65° C. in the column oven. A gradient system consisting of mobile phase A (60:40, v/v) acetonitrile:water containing 10 mM ammonium formate with 0.1% formic acid and mobile phase B (90:10:4, v/v/v) isopropanol:acetonitrile:water containing 10 mM ammonium formate with 0.1% formic acid. The sample analysis was performed over 14 min total run time. The initial starting conditions were 85% A and 15% B, and then stayed for 0.3 min with same gradient. The gradient was ramped to 30% B for 1.7 min, kept for 2 min, increased to 50% B for 0.2 min, increased to 80% B to 9 min. The solvent was increased to 100% B for 0.3 min and held to 11.5 min. Subsequently, the system was switched to the initial ratio for 0.3 min, and equilibrated at the initial ratio for additional 2.2 min. The flow rate was 0.5 mL/min and the injection volume was 5 μL. TOF MS acquisition time was 0.25 seconds, and MS/MS acquisition time was 0.1 seconds. The scan range was m/z 70-1700 for TOF MS and m/z 50-1700 for MS/MS. Source parameters included nebulizing gases GS1 at 45, GS2 at 50, curtain gas at 35, positive mode ion spray voltage 5500 V, negative mode ion spray voltage at −4500 V, declustering potential at 80 and −80 V, and at an ESI source operating temperature of 550° C. Collision energy for MS/MS step was 35±10 eV. Data was imported into PeakView software for relative quantification and identification. Sphingolipids and fatty acids species were confirmed by high resolution MS, MS/MS fragmentation, and isotopic distribution, and then compared using the PeakView database. Sphingolipids, TAG and CHOL were identified in positive ion mode as [M+H]+, fatty acids and CHOL-3-Sulfate in negative ion mode as [M−H]−, respectively.

Statistical Methods

For comparisons between lipid profiles of normal healthy, AD-S. aureus− and AD-S. aureus+ subjects prior to age and gender adjustment, Student's unpaired t-test was performed to analyze statistical significance of differences between groups. Graphics were created with Graph Pad Prism software (Graphpad Software, La Jolla, Calif.).

To determine whether lipid level is associated with S. aureus colonization status and/or TEWL, general linear regression models adjusting for age and sex were used to estimate the mean (or geometric mean for non-Normally distributed outcomes) and associated 95% confidence interval in each AD diagnosis group. The association between lipidomics parameters and measures of barrier function or integrity were also estimated using similar models, although in this case, the mean (or geometric mean) of the lipidomics parameter was estimated for an average participant in our study (specifically, a 41 year-old Female who is AD S. aureus+). Statistical significance was based on a two-sided significance level of 0.05. All p-values reported were considered descriptive. No adjustments for multiple comparisons were made. SAS version 9.4 software (SAS Institute, Inc, Cary, N.C.) was used for all analyses.

### Example 5

### Testing Atopic Dermatitis Amelioration in an Animal Model

Ctip2^{eP-/-} mice, lacking CTIP2 in the epidermis of the skin, provide an animal model of atopic dermatitis.

Ctip2^{eP-/-} mice are provided and allowed to progress until atopic dermatitis symptoms appear. The lipid profile of the skin of the mice is determined and compared to a control, for example using the methods provided herein. Deficiencies in certain lipids are noted and a formulation that includes the deficient lipids is made. The composition is administered to the mice and the response monitored. In some examples, the physical appearance of the skin is monitored. In some examples, a second lipid profile is determined and compared to the first, for example, to determine if the treatment restored the deficient lipids.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

1. A method for determining a lipid imbalance in a subject, comprising:
providing one or more tape strips comprising a skin surface sample obtained from the subject;
extracting epidermal lipids from the skin surface sample;
detecting a composition of lipids present in the extracted sample; and
comparing the composition of lipids to a control, wherein a difference in the composition of lipids as compared to the control identifies the lipid imbalance in the subject.

2. The method of claim 1, wherein extracting epidermal lipids from the skin surface sample comprises contacting the one or more tape strips with an extraction solvent.

3. The method of claim 2, wherein the extraction solvent comprises a mixture of a non-polar solvent, a polar solvent, and water.

4. The method of claim 3, wherein the mixture comprises chloroform (CHCl₃), methanol, and water.

5. The method of claim 4, wherein the ratio of chloroform, methanol, and water is approximately 1:2:0.5, respectively.

6. The method of claim 1, wherein detecting the composition of lipids present in the extracted sample comprises mass spectral analysis and/or chromatography.

7. (canceled)

8. The method of claim 1, further comprising assigning the subject to a skin lipid deficiency category on the basis of the lipid imbalance in the subject.

9. The method of claim 8, wherein the skin lipid deficiency category comprises one of:
a) deficiency category group I, wherein the subject is deficient in one or both of lipids consisting of FFA 16:1 and FFA 18:1;
b) deficiency category group II, wherein the subject is deficient in one or more of lipids consisting of CER[AH]C48, CER[EOH]C66 and CER[EOH]C68; or
c) deficiency category group III, wherein the subject is deficient in one or more of lipids consisting of CER[AP]C40, CER[NDS]C52, CER[NDS]C54, CER[AH]C48 and CER[EOS]C70 lipids.

10. The method of claim 9, further comprising providing a therapeutic composition formulated to ameliorate the lipid present in the skin lipid deficiency category, wherein the composition comprises:
a) FFA 16:1 and FFA 18:1 for deficiency category group I;
b) FFA 16:1, FFA 18:1, CER[AH]C48, CER[EOH]C66 and CER[EOH] C68 for deficiency category group II; or
c) FFA 16:1, FFA 18:1, CER[AP]C40, CER[NDS]C52, CER[NDS]C54, CER[AH]C48, CER[EOH]C66, CER[EOH] C68, and CER[EOS]C70 for deficiency category group III.

11. The method of claim 1, further comprising:
preparing a personalized topical medicament for treating the lipid imbalance; and
providing the personalized topical medicament to the subject.

12. The method of claim 1, wherein the subject has, or is suspected of having, atopic dermatitis, eczema, psoriasis, ichthyosis or Netherton Syndrome or any other skin diseases manifested with barrier disruption and monitored by increased trans epidermal water loss (TEWL).

13. A method of augmenting a lipid deficiency in skin of a subject, comprising:
identifying a deficiency in one or more lipids in a skin sample obtained from the subject;
formulating a topical therapeutic composition that contains the one or more lipids, a similar lipid, or a subset thereof; and
providing the composition to the subject.

14. The method of claim 13, wherein identifying the deficiency in one or more lipids in the skin sample obtained from the subject, comprises:
providing one or more tape strips comprising a skin surface sample obtained from the subject;
extracting epidermal lipids from the skin surface sample;
detecting a composition of lipids present in the extracted sample; and comparing the detected composition of lipids to a control, wherein a reduction in the one or more lipids in the composition of the lipids as compared to the control identifies the deficiency in the one or more lipids.

15. The method of claim 14, wherein extracting epidermal lipids from the skin surface sample comprises contacting the one or more tape strips with an extraction solvent.

16. The method of claim 15, wherein the extraction solvent comprises a mixture of a non-polar solvent, a polar solvent, and water.

17. The method of claim 16, wherein the mixture comprises chloroform (CHCl₃), methanol, and water.

18. The method of claim 17, wherein the ratio of chloroform, methanol, and water is approximately 1:2:0.5.

19. The method of claim 18, wherein detecting the composition of lipids present in the extracted sample comprises mass spectral analysis and/or chromatography.

20. (canceled)

21. The method of claim 13, wherein the subject has, or is suspected of having, atopic dermatitis, eczema, psoriasis, Netherton syndrome, or ichthyosis or any other skin diseases manifested with barrier disruption and monitored by increased trans epidermal water loss (TEWL).

22. The method of claim 13, wherein the method is a method of treating or inhibiting Staphylococcus aureus infection in the skin of the subject.

23. A topical formulation for augmenting a lipid deficiency in the skin of a subject, comprising:
a) FFA 16:1 and FFA 18:1;
b) FFA 16:1, FFA 18:1, CER[AH]C48, CER[EOH]C66 and CER[EOH] C68;
c) FFA 16:1, FFA 18:1, CER[AP]C40, CER[NDS]C52, CER[NDS]C54, CER[AH]C48, CER[EOH]C66, CER[EOH] C68, and CER[EOS]C70;
d) FFA16:1, FFA18:1, TG48:1, TG48:2, TG50:1, TG50:2, TG50:3, TG58:2, CER[AH]C38, and CER[AP]C40;
e) CER[NDS]C52, or CER[NDS]C54;
f) TG46:2, CER[AH]C48, CER[EOH]C66, CER[EOH]C68, and CER[EOS]C70; or
g) CER[AH]C38, CER[AH]C48, CER[AP]C40, CER[NDS]C52, CER[NDS]C54, CER[EOH]C66, CER[EOH]C68, CER[EOS]C70, FFA16:1, FFA18:1, TG46:2, TG48:1, TG48:2, TG50:1, TG50:2, TG50:3, and TG58:2.
